(19) United States
(12) Patent Application Publication
(10) Pub. No.: US 2003/0143634 A1
(43) Pub. Date: Jul. 31, 2003

(54) Method to detect interactions between cellular components in intact living cells, and to extract quantitative information relating to those interactions by fluorescence redistribution

(75) Inventors: Bernard Robert Terry, Frederiksberg (DK); Soren Jensby Nielsen, Frederiksberg (DK)

Correspondence Address: BIRCH STEWART KOLASCH & BIRCH, PO BOX 747, FALLS CHURCH, VA, 22040-0747, US

(73) Assignee: Biolmage A/S, Soborg (DK)

(21) Appl. No.: 10/270,223

(22) Filed: Oct. 11, 2002

Related U.S. Application Data
(63) Continuation of application No. PCT/DK02/00651, filed on Oct. 1, 2002.
(60) Provisional application No. 60/328,896, filed on Oct. 11, 2001.

(30) Foreign Application Priority Data
Oct. 1, 2001 (DK) PA 2001 01433

(51) Int. Cl. G01N 33/53; G01N 33/567; C12N 5/00

(52) U.S. Cl. 435/7.2; 435/325

(57) ABSTRACT

The present invention relates to a 3-part hybrid system for detection protein interactions in live mammalian cells and screening for compounds modulating such interactions. The method is fully compatible with HTS. The three hybrids are a first heterologous conjugate comprising an anchor protein that specifically binds to an internal structure within the cell conjugated to an interactor protein of type A, a second heterologous conjugate comprising an interactor protein of type B conjugated to the first protein of interest, a third heterologous conjugate comprising a second protein of interest conjugated to a detectable group. When applying a dimerizer compound, interactor proteins A and B bind to each other and if the two proteins of interest interact, the distribution of the detectable group will mimic the distribution of the anchor protein. However, if there is no interaction, the distribution of the detectable group will mimic the distribution of the second protein of interest.

### CROSS-REFERENCES TO RELATED APPLICATIONS

This application is a Continuation of co-pending PCT International Application No. PCT/DK02/00651 filed on Oct. 1, 2002, which designated the United States and on which priority is claimed under 35 U.S.C. § 120, the entire contents of which are hereby incorporated by reference.

### FIELD OF INVENTION

The present invention relates to an improved method for measurement of interactions between two components wherein the two components are present in a cell, and where both components are most usually wholly or mainly proteinaceous in composition (i.e. the interaction is a protein-protein interaction, or protein-protein binding event). This is presently referred to as “improved GFP assisted Readout of Interacting Proteins (iGRIP)”

### BACKGROUND OF THE INVENTION

The interaction of proteins with each other and with other cellular components is an intrinsic part of nearly every cellular process, and this is especially true of intracellular signaling systems. Information is passed through and between signaling systems by a series of such interactions. In order to study the function of a protein, a practical strategy is first to identify the components that it interacts with. Most of these will be other proteins—sometimes of the same species, but most often a very different type and with very different functional characteristics.

The identification of novel interactions is a very rapidly growing area of research in cell biology and signal transduction. A notable feature of recent discoveries in this area is the specificity with which partners interact, equaling or exceeding the degree of specificity commonly seen in ligand-receptor interactions seen at the cell surface (Pawson T, Nash P Genes Dev 14:8, 2000). Identification of interacting species brings with it the opportunity to identify novel signaling interactions that may assist greatly in the functional characterization of proteins involved in cellular signaling. In addition, these interactions are applicable to the development of new pharmaceutical agents capable of disrupting or engaging partners in an interaction. Compounds with this mechanism of action will be able to modulate the flow of information through signaling pathways, and in so doing find application in very many areas of human and animal health care (Huang, Z Pharmacol & Toxicol 86, 2000). Since such compounds will be inherently very selective and have their action without the need for gross inhibition of catalytic activity, it can be expected that therapies based on their use will not carry with them the problems of poor specificity and damaging side effects commonly associated with more traditional active-site inhibitors.

Existing methods for identification of interacting species can be divided into two groups: First are those methods that can only work with more or less purified components brought together in vitro, such as surface plasmon resonance (evanescent wave methods), protein mass spectrometry, fluorescence correlation spectroscopy and anisotropy measurements all with the common feature that the components of interest are isolated from the cellular context. The second group includes all methods designed to work within living cells. Of these, many have been developed to work in yeast cells (yeast two hybrid, reverse yeast two hybrid and variations thereof), but some have been adapted for use in mammalian cell systems. Cellular methods for detection of protein interactions have been well reviewed by Mendelsohn, A. R., Brent, R. (1999) (Science 284(5422):1948). Many of these methods are descendants of the conventional two-hybrid methods, more broadly described as complementation methods, where transcriptional activity is initiated by the bringing together of bi-partite transcription factors through the interaction of attached “bait” and “prey” components, while other methods rely on reconstitution of a biochemical function in vivo. Rossi et al. (2000) (Trends in Cell Biology 10:119-122) have thus developed a mammalian cell-based protein-protein interaction assay where the read-out is not transcriptional, but reconstitution of a mutated beta-galactosidase enzyme. Upon reconstitution of the tetrameric enzyme, enzymatic activity can be monitored. In addition, methods for monitoring protein-protein interactions that are based on an optical read-out i.e. fluorescence resonance transfer (FRET), or coincidence analysis (a variant of fluorescence correlation spectroscopy), or fluorescence lifetime changes. The last three categories are more normally applied under simplified in vitro conditions, but attempts are being made to move them into the more complex environment of the living cell. Recently, Tobias Meyer reported (WO00/17221) a method wherein two heterologous conjugates are introduced into a cell. The first heterologous conjugate comprises the first protein of interest conjugated to a detectable group (e.g. GFP). The second heterologous conjugate comprises a second protein of interest conjugated to a protein that specifically binds to an internal structure within the cell upon stimulation with phorbol ester. When the second protein is bound to an internal structure within the cell, with a known distribution, binding between the two proteins of interest can be visualized because the detectable group will be located bound to internal structure within the cell.

Many cellular processes are triggered by induced interaction, or dimerization, of signaling proteins. Of particular use is a small molecule that can be externally applied to act as a dimerizer, to initiate the binding of two components. An example of such a dimerizer is the fungally derived immunosuppressive compound rapamycin, that initiates heterodimerization of FK506 binding protein (FKBP12) to the large PI3-kinase homologue FRAP, also known as mTOR or RAFT (Choi et al. 1996). Similarly, FK506, another immunosuppressive compound, initiates the binding of FKBP12 to calcineurin. Both these compounds have been successfully used as the basis for inducible heterodimerization systems (Ho et al. 1996; Rivera et al. 1996). Commercial systems based on these interactions are now available that provide a means to induce homodimerization or heterodimerization of chosen components upon the application of small molecule derivatives of rapamycin and FK506 (Argent™ kits, ARIAD Pharmaceuticals, Inc., Cambridge, Mass.). The advantage of these kits is that the ligand interfaces of the components they provide have been modified to accept synthetic dimerizers that cannot bind to or dimerize the natural species of interacting components in cells; thus the synthetic dimerizers lack the biological activity of the parent molecules, making them ideal for use in studying signaling processes in living cells.

### SUMMARY OF THE INVENTION

The present application describes, for the first time, the use of a three part system for measuring protein interactions. Construction of 3 probes (example 1) and transfection into the same cell (example 2) wherein the first is an anchor protein linked to FRB*, the second is FKBP linked to protein X, and the third is protein Y linked to a GFP (FIG. 5). The distribution of GFP in a stable cell will be independent of the anchor location. However, as long as X and Y interact, application of AP21967 will cause the GFP signal to redistribute to the location of the anchor. If a compound disrupts the binding of X and Y, the GFP signal will not redistribute to the anchor location (as there is no connection between X and Y anymore). This is exemplified in various configurations: Use of a membrane anchor (src(1-14)) to detect compounds disrupting the binding between SOS1 and GRB2 (example 4) or the binding between the CAD system (example 8) and to detect new interaction partners to protein X (example 5). Using a cytoplasmic anchor (F-actin) to detect compounds disrupting the binding between the CAD system (example 6). Using a nuclear anchor to detect compounds disrupting binding between the CAD system (example 7). The test substance used to disrupt have the same efficacy independently of the anchor used. This system is fully compatible with High-Throughput-Screening (HTS) with acceptable z-factors (examples 6 and 7).

### DETAILED DISCLOSURE

Thus, one aspect of the present invention relates to a method for detecting if a compound disrupts the interaction between two intracellular proteins comprising the steps of:

(a) providing a cell that contains three heterologous conjugates,

a first heterologous conjugate comprising an anchor protein that specifically binds to an internal structure within the cell conjugated to an interactor protein of type A

a second heterologous conjugate comprising an interactor protein of type B conjugated to the first protein of interest

a third heterologous conjugate comprising a second protein of interest conjugated to a detectable group,

(b) inducing interaction of protein of type A with protein of type B through application of a dimerizer molecule;

(c) detecting the intracellular distribution of the detectable group

an intracellular distribution of said detectable group mimicking the intracellular distribution of the anchor-protein being indicative of binding between the two proteins of interest;

(d) repeating step (c) with and without the compound;

a change in intracellular distribution of the detectable group with and without the compound being indicative of the compound modulating the interaction between the first and the second protein of interest.

A second closely related aspect relates to a method for detecting if a compound induces interaction between two intracellular proteins comprising the steps of:

(a) providing a cell that contains three heterologous conjugates,

a first heterologous conjugate comprising an anchor protein that specifically binds to an internal structure within the cell conjugated to an interactor protein of type A

a second heterologous conjugate comprising an interactor protein of type B conjugated to the first protein of interest

a third heterologous conjugate comprising a second protein of interest conjugated to a detectable group,

(b) inducing interaction of protein of type A with protein of type B through application of a dimerizer molecule;

(c) detecting the intracellular distribution of the detectable group

an intracellular distribution of said detectable group mimicking the intracellular distribution of the anchor-protein being indicative of binding between the two proteins of interest;

(d) repeating step (c) with and without the compound;

a change in intracellular distribution of the detectable group with and without the compound being indicative of the compound modulating the interaction between the first and the second protein of interest.

The present application details how the knowledge that any particular anchor component is confined to a certain cellular location may be used to explore interactions between intracellular components. If a component is known to be confined to a known cellular location, an interactor protein of class A may be covalently attached to the first component (the “anchor”) and will be expected to assume the same location in the cell as the anchor component to which it is attached. The anchor and class A interactor comprise the first conjugate molecule.

Into the same cell is introduced a second conjugate, that bears an interactor protein of class B covalently attached to the first protein of interest (the “bait” component of a bait-prey pair). When the dimerizer molecule is introduced, the second conjugate is expected to join to the first conjugate molecule, and assume the same location in the cell as the anchor component used in the system.

A third conjugate molecule is introduced into the same cell. The third conjugate bears the second protein of interest (the “prey” component) covalently linked to a labeling molecule that will allow the location of the third conjugate to be detected and measured within the cell. If an interaction occurs between bait and prey, then the prey component also takes up the same distribution within the cell as the [anchor-interactor A]:[interactor B-bait] components, but only if the appropriate dimerizer stimulus has also been applied. Using this system, therefore, makes it possible to distinguish between specific bait-prey interactions and any other condition affecting the distribution of the detectable prey component, see FIG. 5.

Therefore, the 3-part iGRIP method is able to impose a gross redistribution upon interacting components within the cell, even if the components in isolation would not normally display an appreciable redistribution as part of their functional cycle. The method also allows for targeting interactions of different locations within the cell with the purpose of studying whether location specific conditions are necessary for the interaction to occur.

The fact that the interactions measured are in the complex environment of the cell, that allows for the influence of factors that may modulate an interaction in the same way as would happen in the native system, adds important physiological relevance-to the method. Mammalian cell assays provide physiologically relevant context for interactions, to allow or the influence of factors that may modulate an interaction as they would in the native system, for instance as in cells in the intact human system. This is because the method uses no treatments nor conditions that will necessarily affect normal biological processes, including signaling processes, in the living cell.

The compounds have to penetrate the cells and have to survive in the cell for the period of the assay. Thus a response is an indication of bioavailability and the stability of the compound.

As will be illustrated in detail below the response of any of the assays based on the method of the present invention can be monitored either continuously as a sequential series of measurements over time, to generate a time course for a response, or by single end-point measurement. Time course measurements require live cells throughout. End-point measurements can be made on either live or chemically fixed cells.

The present invention includes as anchor components for the method any and all genetically encodable cellular components that have a defined cellular distribution. This is possible by the inclusion of the third component (the first heterologous conjugate) that provides an inducible interaction interface.

Anchor systems can be designed to achieve redistribution to compartments or locations within cells where the proteins of interest will experience the influences that would normally be required to modulate the interaction between those proteins. As an example, some proteins normally require to be phosphorylated or dephosphorylated by enzymes sequestered in the plane of the plasma membrane—for such proteins of interest it is appropriate to choose an anchor component that would be expected to be confined to the plasma membrane, to allow the interacting proteins to be appropriately modified. Thus, in one embodiment, a preferred anchor component that will target the anchor conjugate to the plasma membrane is a protein containing the transmembrane domain of the epidermal growth factor receptor (EGFR), or containing the transmembrane domain of a protein from the integrin protein family, or containing the myristoylation sequence from c-Src (residues 1-14).

In another embodiment, a histone protein is used as the anchor, or a protein normally restricted to nucleoli, for example the p120 nucleolar protein, in order to direct the anchoring conjugate to the nucleus.

In another embodiment, the anchor protein is chosen from those proteins normally confined to mitochondrial outer or inner membranes for example VDAC, F₀ subunit of ATP-ase, or NADH dehydrogenase. In another embodiment, the anchor protein is chosen from the group of proteins normally confined to the various different regions of Golgi bodies for example TGN38 or ADAM12-L. In another embodiment, the anchor protein is chosen from the group of proteins normally confined to focal adhesion complexes for example P125, FAK, integerin alpha or beta, or paxillin. In another embodiment, the anchor protein is chosen from the group of proteins normally associated with cytoskeletal structures such as F-actin strands or micro tubular bundles for example MAP4, actin binding domain of alpha-actinin (actinPaint), kinesins, myosins or dyniens.

The particular utility of stimulus-induced redistributions, such as those that are based on the use of dimerizer molecules, is that in one and the same cell it is possible to switch on a distinctive distribution where previously there was none. This not only guarantees, in advance, that the distinctive distribution is purely a result of specific interaction between the bait and prey components, but also guarantees that this interaction will give a signal that is measurable by the assay equipment configured to detect the specific and expected distribution of the anchor component. In effect, this latter point means that many different bait-prey interactions can be measured and assayed for any particular anchor component without the need to reconfigure the measuring equipment or the assay method. Also, the dimerization stimulus becomes a reference compound in screening assays by which the maximum and minimum expected signals for an assay can be determined. Thus, it is crucial that interactor A and interactor B have no measurable affinity for each other in the absence of a dimerizer compound. In one embodiment therefore, interactors A and B are chosen in appropriate pairs among the proteins targeted by immunosuppressants such as, but not limited to, cyclosporin A, Rapamycin and FK506. These proteins include, but are not limited to, FKBP12, FRAP and cyclophilin. In a preferred embodiment, interactors A and B are represented by FKBP12 and a mutated fragment of FRAP (FRB T2098L, ARIAD Pharmaceuticals) and the dimerizer is represented by AP21967 (ARIAD Pharmaceuticals).

In another embodiment, the ligand-binding domain of a steroid hormone receptor such as, but not limited to, the estrogen receptor is used as both interactor A and B. Such a ligand-binding domain will homo-dimerize upon addition of its cognate hormone ligand (in this case estrogen).

In another embodiment, the full-length or the ligand-binding domain of a steroid hormone receptor is chosen as interactor A whereas interactor B is chosen among the family of steroid hormone receptor co-activators including, but not limited to, SRC-1, GRIP-1, ACTR, AIB-1. As above, cognate hormone is used as dimerizer molecule.

Thus, a specific embodiment of the present invention relates to a method wherein the application of a specific dimerizer stimulus redistributes the chosen bait-prey pair to any chosen and defined location within the cell, and where the dimerizer stimulus by itself has no ability to stimulate or inhibit inherent signaling activity within the cell of interest. In a preferred embodiment, the dimerizer stimulus is fully reversible, and a competitive reference compound is known, also without biological activity in the cell, that can be used to compete for binding of the dimerizer compound to one or both of the ligand interfaces of interactor components of class A and class B. Binding of the competitive reference compound reverses the dimerization (see example 7, FIG. 22).

Similarly, it is possible to use any constitutively interacting protein pair if a known compound exists that will inhibit the dimerization of the protein pair. By using the specific protein pair in question as interactors of class A and B in the method outlined above and by either measuring loss of specific (anchor-like) localization of the detectable probe upon addition of interaction inhibitor, or by measuring gain of specific (anchor-like) localization of the detectable probe upon withdrawal of interaction inhibitor, the two methods become identical. Thus, withdrawal of A-B interaction inhibitor is identical to addition of A-B dimerizer, and addition of A-B interaction inhibitor is identical to withdrawal of A-B dimerizer.

Thus, one embodiment of the invention uses the constitutively homodimerizing F36M mutant of FKBP12 (ARIAD Pharmaceuticals) as interactors A and B and FKBP12 ligands including, but not limited to, FK506 and Rapamycin as inhibitors of the A-B interaction.

In another embodiment, FKBP12 and the type I TGF-beta receptor are chosen as interactors A and B and FKBP12 ligands including, but not limited to, FK506 and Rapamycin as A-B inhibitors.

An example of suitable combinations of linker protein A, linker protein B, dimerizer compound and competitive reference compound are listed in Table 1.

TABLE 1
Suitable combinations of linkers and compounds

| linker | linker | dimerizer | competitive |
|---|---|---|---|
| protein A | protein B | compound | reference compound |
| FKBP12 | FRAP | Rapamycin | FK506 |
| FKBP12 | FRB (T2098L) | Rapamycin | FK506 |
| FKBP12 | FRB (T2098L) | AP21967 | FK506 |
| FKBP12 | FKBP12 | AP21967 | FK506 |
| FKBP12 | FKBP12 | AP21967 | Rapamycin |
| FKBP12 | Calcineurin | FK506 | Rapamycin |
| Cyclophilin | Calcineurin | Cyclosporin | |

In yet another embodiment, a full-length or ligand-binding domain only nuclear hormone receptor including, but not limited to, the thyroid hormone receptor or the retinoid acid receptor is chosen as interactor A and a full-length (or fragment thereof) nuclear hormone co-repressor such as, but not limited to, N-CoR or SMRT as interactor B. In this case cognate hormone is used as dimerization inhibitor.

One particular advantage of the present method is that it allows for counter screens. That is, to test if the compounds identified are true modulators of the interaction between the two proteins of interest, or if they modulate either the interaction between protein of type A and protein of type B, or directly or indirectly affect the location of the anchor component. Thus, in one embodiment the screening method further comprises a counter screen comprising the steps of:

(i) providing a cell that contain two heterologous conjugates,

a first heterologous conjugate comprising an anchor protein that specifically binds to an internal structure within the cell conjugated to an interactor protein of type A

a second heterologous conjugate comprising an interactor protein of type B conjugated to a detectable group

(ii) inducing interaction of protein of type A with protein of type B through application of a dimerizer molecule;

(iii) detecting the intracellular distribution of the detectable group

an intracellular distribution of said detectable group mimicking the intracellular distribution of the anchor-protein being indicative of binding between the protein of type A and the protein of type B

(iv) repeating step (iii) with and without the compound found to disrupt the binding between the two proteins of interest;

a change in intracellular distribution of the detectable group with and without said compound found to disrupt the binding between the two proteins of interest being indicative that the compound is a false positive capable of disrupting the binding between protein of type A and protein of type B.

When designing the counter screen above, the same interactor proteins of type A and type B is used that comprise the dimerizer induced link between mediator and anchor conjugates in the original screen. The anchor used, is preferably the same as the anchor used in the original screen. However, sometimes using a different anchor, and performing two counter screens, will provide more specific information about the nature of interfering (false positive) compounds.

In general, the dimerizer stimulus can be applied either before or after any interaction has occurred between bait and prey components. Therefore, the location and environment in which an inducible or transient interaction takes place can be controlled.

It is preferred that the dimerization process between class A and class B components under the control of the dimerizer compound is rapid (detectable within several minutes), as compared to many other cellular systems that report on protein interactions, that include for example transcriptional reporter systems or reconstitution of enzymes and subsequent assay of their activity.

It is anticipated that in certain instances, the interaction between protein X and protein Y, the two proteins of interest, requires a further specific “interaction stimulus”. Thus, they may not interact until stimulated to do so. This justified the ability of the system to relocate a component to the relevant compartment. An example of such system is the IRS-1. IRS-1 needs tyrosine phosphorylation before it will interact with Grb2. Such phosphorylation is carried.out by the insulin receptor located in the plasma membrane. The interaction stimulus in that system could then be Insulin.

The term “compound” is intended to indicate any sample, that has a biological function or exerts a biological effect in a cellular system. The sample may be a sample of a biological material such as a sample of a body fluid including blood, plasma, saliva, milk, urine, or a microbial or plant extract, an environmental sample containing pollutants including heavy metals or toxins, or it may be a sample containing a compound or mixture of compounds prepared by organic synthesis or genetic techniques. The compound may be small organic compounds or biopolymers, including proteins and peptides.

Numerous cell systems for transfection exist. A few examples are Xenopus oocytes or insect cells, such as the sf9 cell line, or mammalian cells isolated directly from tissues or organs taken from healthy or diseased animals (primary cells), or transformed mammalian cells capable of indefinite replication under cell culture conditions (cell lines). However, it is preferred that the cells used are mammalian cells. This is due to the complex biochemical interactions specific for each cell type. The term “mammalian cell” is intended to indicate any living cell of mammalian origin. The cell may be an established cell line, many of which are available from The American Type Culture Collection (ATCC, Virginia, USA) or similar Cell Culture Collections. The cell may be a primary cell with a limited life span derived from a mammalian tissue, including tissues derived from a transgenic animal, or a newly established immortal cell line derived from a mammalian tissue including transgenic tissues, or a hybrid cell or cell line derived by fusing different cell types of mammalian origin e.g. hybridoma cell lines. The cells may optionally express one or more non-native gene products, e.g. receptors, enzymes, enzyme substrates, prior to or in addition to the fluorescent probe. Preferred cell lines include, but are not limited to, those of fibroblast origin, e.g. BHK, CHO, BALB, NIH-3T3 or of endothelial origin, e.g. HUVEC, BAE (bovine artery endothelial), CPAE (cow pulmonary artery endothelial), HLMVEC (human lung micro vascular endothelial cells), or of airway epithelial origin, e.g. BEAS-2B, or of pancreatic origin, e.g. RIN, INS-1, MIN6, bTC3, aTC6, bTC6, HIT, or of hematopoietic origin, e.g. primary isolated human monocytes, macrophages, neutrophils, basophils, eosinophils and lymphocyte populations, AML-14, AML-193, HL-60, RBL-1, U937, RAW, JAWS, or of adipocyte origin, e.g. 3T3-L1, human pre-adipocytes, or of neuroendocrine origin, e.g. AtT20, PC12, GH3, muscle origin, e.g. SKMC, A10, C2C12, renal origin, e.g. HEK 293, LLC-PK1, or of neuronal origin, e.g. SK-N-DZ, SK-N-BE(2), HCN-1A, NT2/D1, or U2-OS of human osteo-sarcoma origin.

The examples of the present invention are based on CHO cells. Therefore, fibroblast derived cell lines such as BALB, NIH-3T3 and BHK cells are preferred.

It is preferred that the three heterologous conjugates are introduced into the cell as plasmids, e.g. three individual plasmids mixed upon application to cells with a suitable transfection agent such as FuGENE so that transfected cells express and integrate all three heterologous conjugates simultaneously. Plasmids coding for each conjugate will contain a different genetic resistance marker to allow selection of cells expressing those conjugates. It is also preferred that each of the anchor and second conjugates also contains a distinct amino acid sequence, such as the HA or myc or Flag markers, that may be detected immunocytochemically so that the expression of these conjugates in cells may be readily confirmed. The third conjugate is already detectable since it bears the detectable group (preferably a green fluorescent protein, GFP) required by the method. Many other means for introduction of one or both of the conjugates are evenly feasible e.g. electroporation, calcium phosphate precipitate, microinjection, adenovirus and retroviral methods, bicistronic plasmids encoding both conjugates etc.

In another embodiment, it is preferred that the conjugate containing the chosen anchor protein is first transfected into cells, and that these cells are then put under selection pressure appropriate to the genetic resistance marker included in the construction of that plasmid, in order to select cells stably expressing the anchor conjugate. Individual clonal cell lines are further sub-selected from the population of cells stably expressing the anchor conjugate in order to establish lines with homogenous properties of expression level and location of the anchor conjugate.

Clonal anchor conjugate lines are then transfected with the second conjugate, bearing the first bait molecule of interest. Again, these cells are put under selection pressure appropriate to the two different genetic resistance markers included in the construction of the anchor conjugate and second conjugate plasmids, in,~order to select cells stably expressing both conjugates. Individual clonal cell lines are further sub-selected from the population of cells stably expressing both conjugates in order to establish lines with homogenous properties of expression level and location of those conjugates.

Cells stably expressing anchor and second conjugates are then separately transfected with plasmid coding for a third (detectable) conjugate. These can then be screened for redistribution behavior in response to the dimerizer stimulus either during the transient phase of expression, or after they have undergone selection for stable expression.

The procedure of separately transfecting cells with each of the three required conjugates, so that a stable and clonal line is first established expressing the anchor conjugate, that is then transfected with to produce clonal lines stably expressing an additional (second) conjugate, is the preferred method for screening cDNA libraries for protein partners to a given bait component. The particular advantage of this procedure is that the location and behavior of the anchor conjugate is defined and established prior to introduction of any further conjugates. When the second conjugate is introduced, that bears the bait protein of interest, its response to the dimerizer stimulus can be tested and defined prior to introduction of any third conjugates. The final step of introducing the third conjugate into such cells, can be performed in parallel with many different types of third conjugate, each bearing different potential prey components. A library of such prey components inserted as detectable conjugates can be readily assembled by one skilled in the art from any cDNA library. A detection vector that forms the basis of a library of detectable conjugates can be assembled by standard DNA cloning techniques. The cDNA inserts can be transferred into the detection vector by restriction enzyme digestion-ligation, by polymerase chain reaction techniques or by recombinase techniques such as those provided by the Gateway™ system (Invitrogen). The various components are illustrated in FIG. 1, FIG. 2, and FIG. 3.

Thus, one aspect of the present invention relates to a method for identifying novel interaction partners for a bait protein comprising the steps of:

(a) providing a cell line where each cell contains two heterologous conjugates,

the first heterologous conjugate comprising an anchor protein that can specifically bind to an internal structure within the cell conjugated to an interactor protein of class A

the second heterologous conjugate comprising an interactor protein of class B conjugated to the bait protein

(b) introducing into said cell line a cDNA library coding for prey proteins conjugated to a detectable group

(c) detecting the intracellular distribution of the detectable group

(d) inducing interaction of protein of type A with protein of type B through application of a dimerizer molecule

(e) detecting the intracellular distribution of the detectable group

the intracellular distribution of said detectable group mimicking the intracellular distribution of the anchor-protein only in the presence of said dimerizer molecule being indicative of binding between the bait and prey proteins

(f) isolating prey conjugates that show indication of binding to the bait component.

In one embodiment of this invention, the cDNA library is produced as an ordered collection and introduced into the bait cell line by High Throughput transfection using techniques such as those developed by Xantos Biomedicine AG (Martinsried, Germany). This has the added advantage of facilitating the identification of positives from the screen.

In another embodiment, the cDNA library is introduced into the bait cell line by transfection followed by selection, such as by fluorescence associated cell sorting or FACS, for those cells that express the detectable group. The expressing cells are then exposed to a reagent that specifically quenches anchor-like signals and those cells that retain signal are selected for further analysis. In the case of a membrane-located anchor and a fluorescent detection group such as GFP, a membrane-specific fluorescence quencher such as acid red can be used (for details see WO 01/81917). This strategy has the added advantage of reducing the number of false positives in the screen.

In yet another embodiment the cDNA library already exists as an ordered collection of stable cell lines, each expressing one prey-detectable group fusion protein. The interaction assay is then performed by adding cells from the bait cell line into wells containing the prey cell line collection and fusing the cells using PEG-mediated cell fusion (StGroth and Scheidegger, 1980). Optionally, cell hybrids containing both a bait and a prey protein can be selected for using different resistance markers on the bait and prey plasmids.

Once an interacting bait-prey pair of components has been identified in an iGRIP cell line, it is a straightforward process to create from that very same cell line a cellular assay compatible with high throughput screening (HTS) methods. This assay may be used to find compounds that will modulate the interaction of the bait and prey components. Once interaction (conditional or constitutive) between the bait and prey components has been demonstrated through appropriate dimerizer-induced redistribution of fluorescence in the cell line, it may be necessary to select responding cells from the background population of non-responding cells in order to achieve an assay cell line with an homogenous and robust response suitable for HTS. Selection of responding cells to create the HTS assay line may be achieved by single cell cloning methods, or by fluorescence activated cell sorting (FACS) methods.

Throughout the present invention, the term “protein” should have the general meaning. That includes not only a translated protein, or protein fragment, but also chemically synthesized proteins. For proteins translated within the cell, the naturally, or induced, post-translational modifications such as glycosylation and lipidation are expected to occur and those products are still considered proteins.

The term intracellular protein interaction has the general meaning of an interaction between two proteins, as described above, within the same cell. The interaction is due to covalent and/or non-covalent forces between the protein components, most usually between one or more regions or domains on each protein whose physico-chemical properties allow for a more or less specific recognition and subsequent interaction between the two protein components involved. In a preferred embodiment, the intracellular interaction is a protein-protein binding.

The detectable group of the third conjugate allows the spatial distribution of that conjugate to be visualized and measured. In a preferred aspect of the invention, the detectable group is a luminophore capable of being redistributed in substantially the same manner as the second protein of interest. In yet another embodiment of the invention, the luminophore is capable of being quenched upon spatial association with a component that is also redistributed by the dimerizer stimulus, or by modulation of some signaling pathway, the quenching being measured as a change in the intensity or lifetime of the luminescence.

In a preferred aspect, the luminophore is a fluorophore. In a preferred embodiment of the invention, the luminophore is a polypeptide encoded by and expressed from a nucleotide sequence harbored in the cell or cells. For example the luminophore is a part of a hybrid polypeptide comprising a fusion of at least a portion of each of two polypeptides one of which comprises a luminescent polypeptide and the other one of which comprises the bait component. Examples of fluorescent proteins are AmCyan, ZsGreen, ZsYellow, DsRed, AsRed and HcRed. They are derived form the phylum of coelenterata and belong to the class of Anthozoa, reef corals. As the examples are carried out with GFP, GFP is especially preferred as the luminophore. The GFP is N- or C-terminally tagged, optionally via a peptide linker, to the biologically active polypeptide or a part or a subunit thereof.

In the present context, the term “green fluorescent protein” (GFP) is intended to indicate a protein that, when expressed by a cell, emits fluorescence upon exposure to light of the correct excitation wavelength (e.g. as described by Chalfie, M. et al. (1994) Science 263, 802-805). Such a fluorescent protein in which one or more amino acids have been substituted, inserted or deleted is also termed “GFP”. “GFP” as used herein includes wild-type GFP derived from the jelly fish Aequorea Victoria, or from other members of the Coelenterata, such as the red fluorescent protein from Discosoma sp. (Matz, M. V. et al. 1999, Nature Biotechnology 17: 969-973), GFP from Renilla reniformis, GFP from Renilla Muelleri or fluorescent proteins from other animals, fungi or plants, and modifications of GFP, such as the blue fluorescent variant of GFP disclosed by Heim et al. (Heim, R. et al., 1994, Proc.Natl.Acad.Sci. 91:26, pp 12501-12504), and other modifications that change the spectral properties of the GFP fluorescence, or modifications that exhibit increased fluorescence when expressed in cells at a temperature above about 30° C. described in PCT/DK96/00051, published as WO 97/11094 on Mar. 27, 1997, and that comprises a fluorescent protein derived from Aequorea Green Fluorescent Protein or any functional analogue thereof, wherein the amino acid in position 1 upstream from the chromophore has been mutated to provide an increase of fluorescence intensity when the fluorescent protein of the invention is expressed in cells. Preferred GFP variants are F64L-GFP, F64L-Y66H-GFP F64L-S65T-GFP, F64L-E222G-GFP. One especially preferred variant of GFP for use in all the aspects of this invention is EGFP (DNA encoding EGFP that is a F64L-S65T variant with codons optimized for expression in mammalian cells is available from Clontech, Palo Alto, plasmids containing the EGFP DNA sequence, cf. GenBank Acc. Nos. U55762, U55763). Another especially preferred variant of GFP is F64L-E222G-GFP.

A specific advantage of using GFP as the detectable group is the non-destructive fluorescence imaging, meaning that the cells can be live and active while being monitored, and since it is based on non-disturbing treatment with the dimerizer molecule, the iGRIP also allows transient or conditional interactions to be monitored. Transient or conditional interactions may occur when components are phosphorylated or otherwise modified during their cycle of operation (e.g. transmission of a signal), and such modifications are common amongst components of intracellular signaling pathways. As the method does not rely on covalent interactions nor on the fact that the components need to have a specific orientation upon interaction, the method is very sensitive and allow for measurement of even low affinity interactions.

Thus, the iGRIP method utilizing GFP as the detectable group makes use of the fact that many cellular components within the cell are confined to specific locations. If those components can be labeled in some way to make them visible in the cell, their location can be measured by a number of image-based techniques. Since imaging techniques are non-destructive, they allow measurements to be made on living cells, hence active processes can be followed over time if that is required—as may be the case when transient events need to be monitored.

In an alternative embodiment, the detectable group is labeled with chemical fluorophores either in situ or by microinjection or otherwise introduced into cells. In yet another embodiment, the detectable group comprises an epitope for antibodies, that are themselves detectable by other methods, either because they are tagged with a fluorophore, or may be detected by a biotin-streptavidin labeling method, or by secondary antibodies labeled with fluorophores etc. Examples of such epitopes are the myc or flag antigens.

Internal cellular structure as used herein refers to a separate, discreet, identifiable component contained within a cell. Such internal structures are, in general, anatomical structures of the cell in which they are contained. Examples of internal structures include both structures located in the cytosol or cytoplasm outside of the nucleus (also called cytoplasmic structures) and structures located within the nucleus (nuclear structures). The nucleus itself including the nuclear membrane is an internal structure.

The recording of the detectable group will vary with the detectable group chosen. For example, when GFP is used as a detectable group the emitted light can be measured with various apparatus known to the person skilled in the art. Typically, such apparatus comprises the following components: (a) a light source, (b) a method for selecting the wavelength(s) of light from the source that will excite the luminescence of the luminophore, (c) a device that can rapidly block or pass the excitation light into the rest of the system, (d) a series of optical elements for conveying the excitation light to the specimen, collecting the emitted fluorescence in a spatially resolved fashion, and forming an image from this fluorescence emission (or another type of intensity map relevant to the method of detection and measurement), (e) a bench or stand that holds the container of the cells being measured in a predetermined geometry with respect to the series of optical elements, (f) a detector to record the light intensity, preferably in the form of an image, (g) a computer or electronic system and associated software to acquire and store the recorded information and/or images, and to compute the degree of redistribution from the recorded images.

In a preferred embodiment of the invention, the apparatus system is automated. In one embodiment, the components in (d) and (e) mentioned above comprise a fluorescence microscope. In one embodiment, the component in (f) mentioned above is a CCD camera. In one embodiment, the component in (f) mentioned above is an array of photo multiplier tubes/devices.

While the stepwise procedure necessary to reduce the image or images to the value representative of the response is particular to each assay, the individual steps are generally well-known methods of image processing. Some examples of the individual steps are point operations such as subtraction, ratioing, and thresholding, digital filtering methods such as smoothing, sharpening, and edge detection, spatial frequency methods such as Fourier filtering, image cross-correlation and image autocorrelation, object finding and classification (blob analysis), and color space manipulations for visualization. In addition to the algorithmic procedures, heuristic methods such as neural networks may also be used.

In one embodiment of the invention, the actual fluorescence measurements are made in a standard type of fluorometer for plates of micro titer type (fluorescence plate reader).

In one embodiment, the optical scanning system is used to illuminate the bottom of a plate of micro titer type so that a time-resolved recording of changes in luminescence or fluorescence can be made from all spatial limitations simultaneously.

In one embodiment, the image is formed and recorded by an optical scanning system.

In a preferred embodiment, the actual luminescence or fluorescence measurements are made in a FLIPR™ instrument, commercially available from Molecular Devices, Inc. Details of such procedure is described in WO00/23615.

In another preferred embodiment, the actual luminescence or fluorescence measurements are made in an evanescent field described in detail in WO00/20859.

The measurement of protein interactions described above is ideal for identification/screening of compounds modulating such interactions. A method by which to carry out such assays, and other assays in High Throughput, as described in WO 02/03072 in summary the method comprises:

(a) contacting or incubating cells to be tested with and without the compound;

(b) adding extraction buffer to the cells of step (a), the extraction buffer comprising a cellular fixation agent and a cellular permeabilization agent; and

(c) measuring the light emitted from the luminophore from cells of step (b);

wherein a difference between light emitted from the cells with and without the influence indicates a difference in the mobility of the cellular component caused by the influence.

The principle is to remove freely mobile luminophore-coupled conjugate from the cell, leaving in place any substantially immobile form of the conjugate.

One major advantage of the extraction procedure is that changes in mobility can be measured as a change in light intensity. As described in the examples, this technique allows Redistribution™ to be detected as a fluorescence intensity change.

A variety of instruments exist to measure light intensity. In a preferred aspect of the present invention, wherein the luminophore is GFP, the instrument for measuring the light emitted from the luminophore is a FLIPR™ (Molecular Devices). In an alternative embodiment, the light emitted from the luminophore is measured on a plate reader.

Based on these scientific and intellectual findings, the present invention can among other things be useful to:

Create cellular assays to monitor interactions between cellular components at the intermolecular level.

Create cellular assays to monitor isolated domains of transient interactions between cellular components.

Create cellular assays to monitor conditional interactions between cellular components.

Create cellular assays to monitor interactions between components that have low affinity for one another.

Create cellular test systems in which the mobility of specific molecular components, for example a species of signaling molecule, can conditionally be restricted (locked down) to achieve a functional knockout of activity for that species.

Create cellular test systems in which the mobility of specific molecular components, for example a species of signaling molecule, can conditionally be released (dispersed) to achieve a functional knock-in of activity for that species.

Create a cellular system where interaction events between specific components are restricted to a specific location

Create cellular assays to find inhibitors of interactions.

Create cellular assays to find activators of interactions.

Create cellular assays to identify novel binding partners to any specific cellular component through screening of cDNA libraries.

Create cellular test systems to investigate the function of specific cellular components.

Create cellular assays to identify the signaling pathways used by orphan receptors and/or orphan ligands.

Create cellular assays in High Throughput screening for interaction modulators.

### LEGENDS TO FIGURES

FIG. 1:

Components of the iGRIP.

The anchor conjugate comprises an anchor protein fused in frame to a linker protein “A”. The 2^{nd} conjugate comprises linker protein “B” fused in frame to the first protein of interest (protein X).

The detectable conjugate comprises the second protein of interest (protein Y) fused in frame to the detectable group, e.g. GFP.

The dimerizer molecule is capable of associating linker protein A with linker protein B.

FIG. 2:

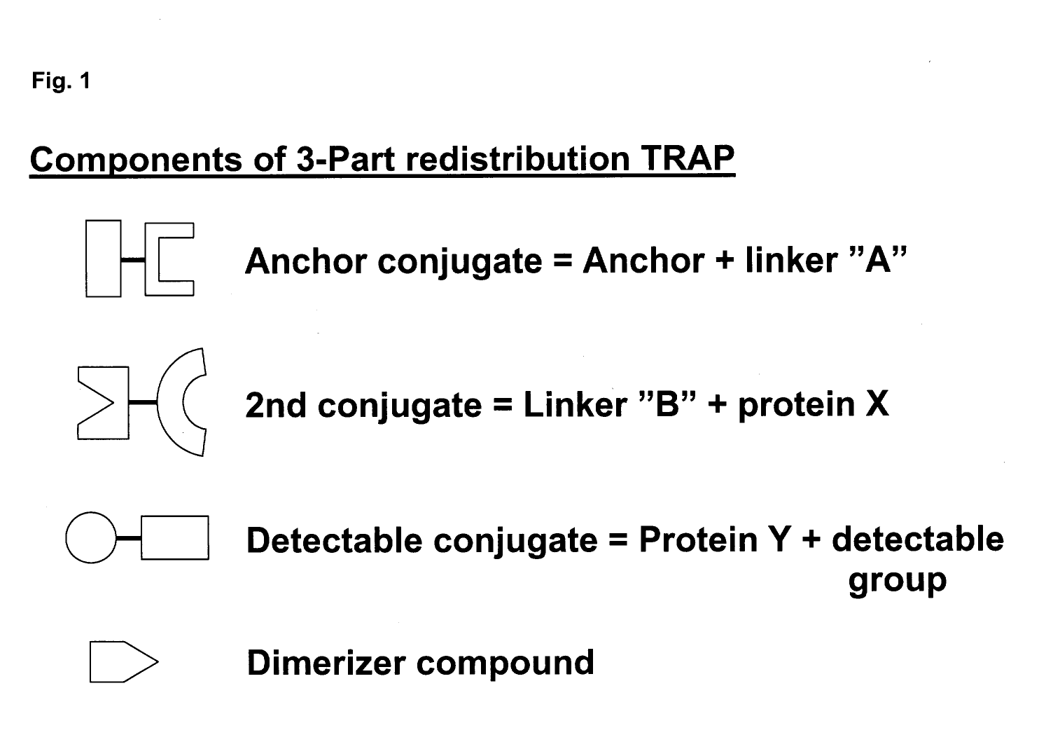

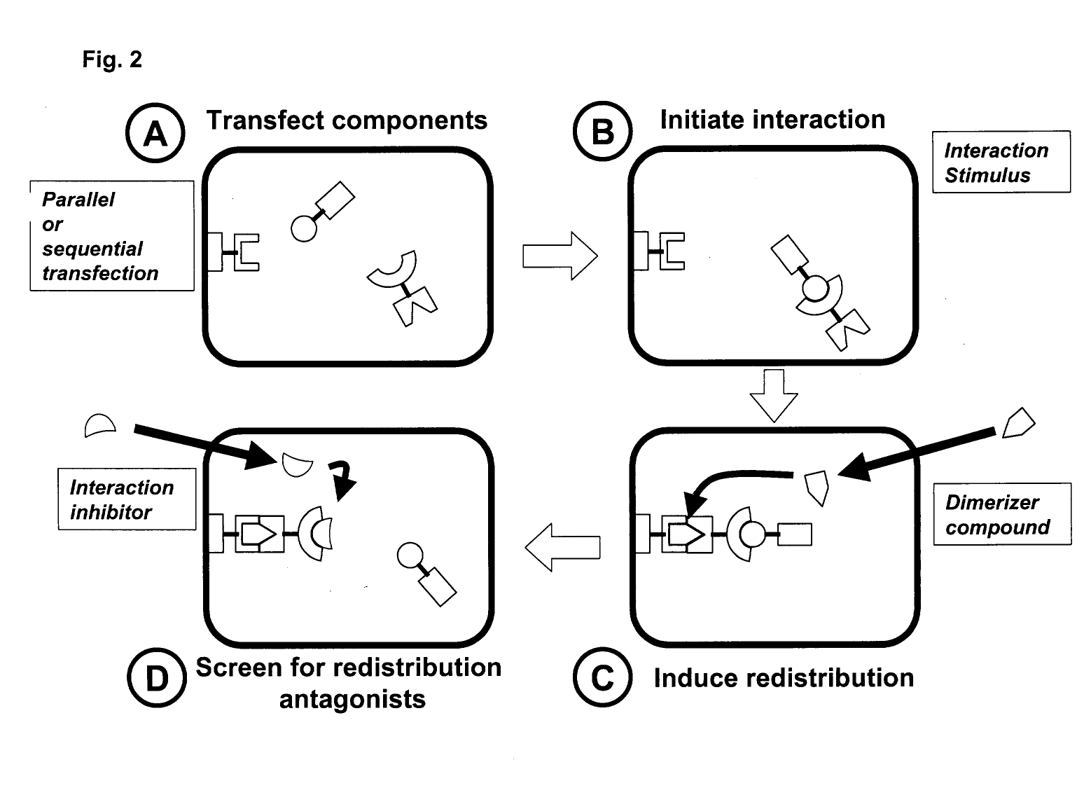

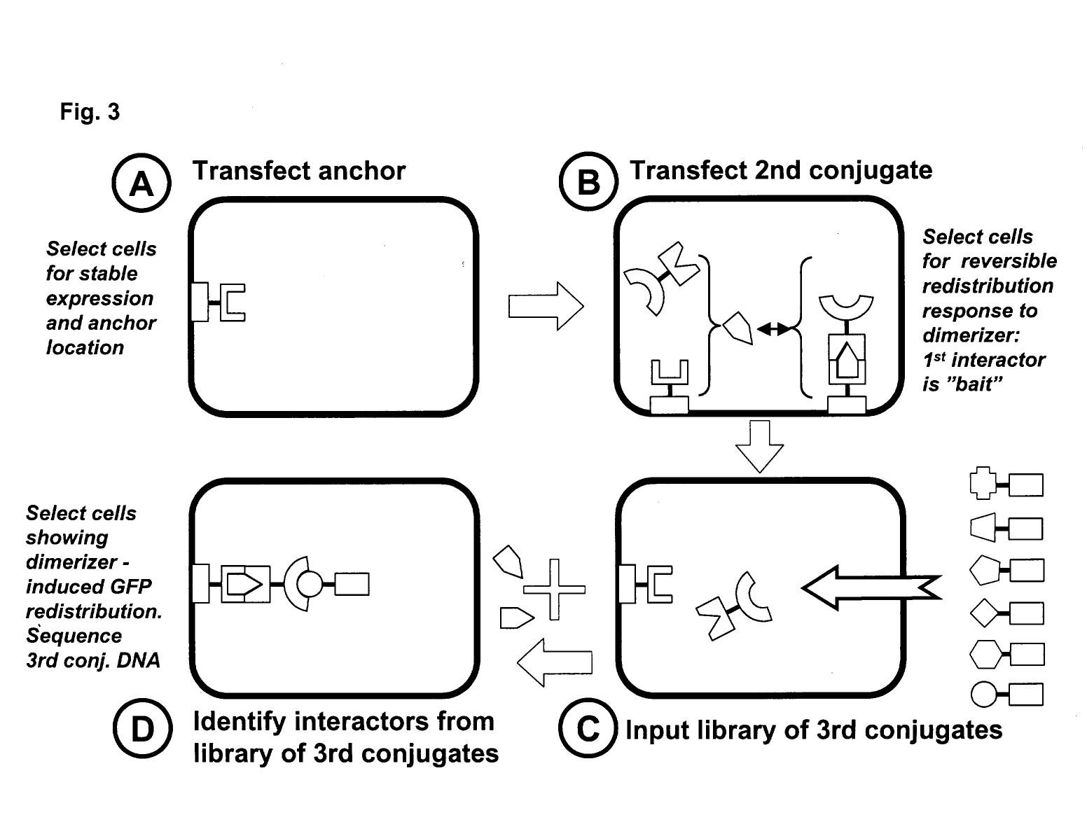

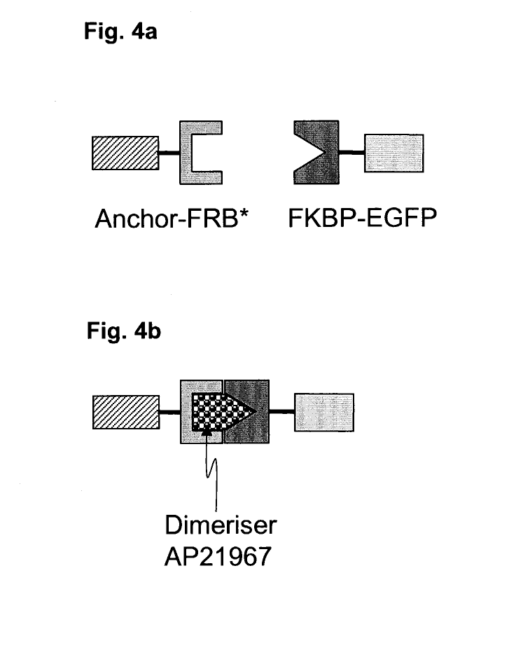

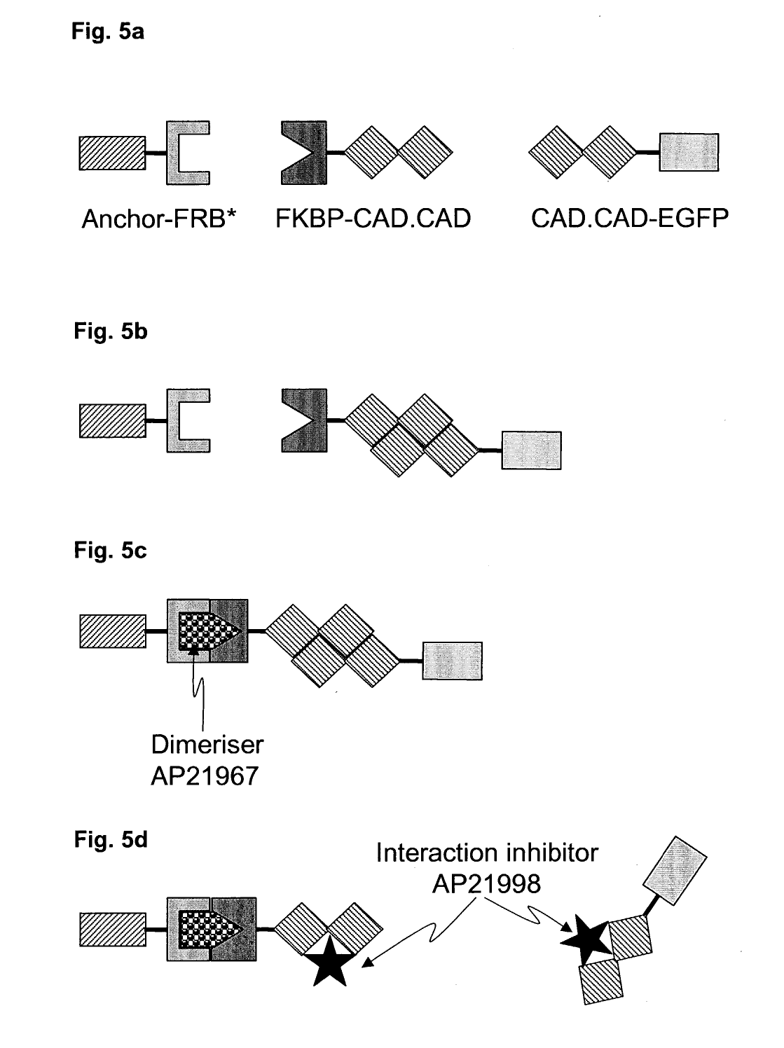

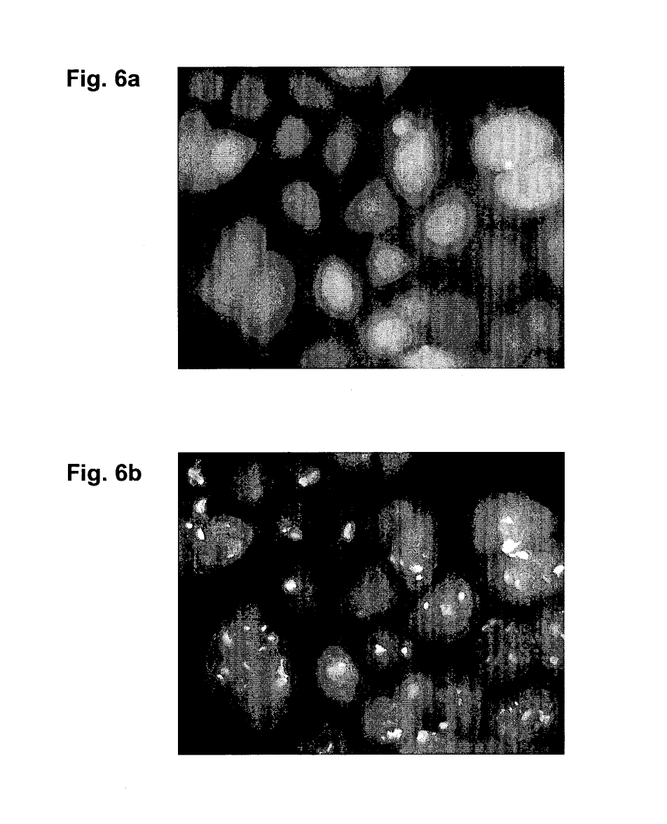

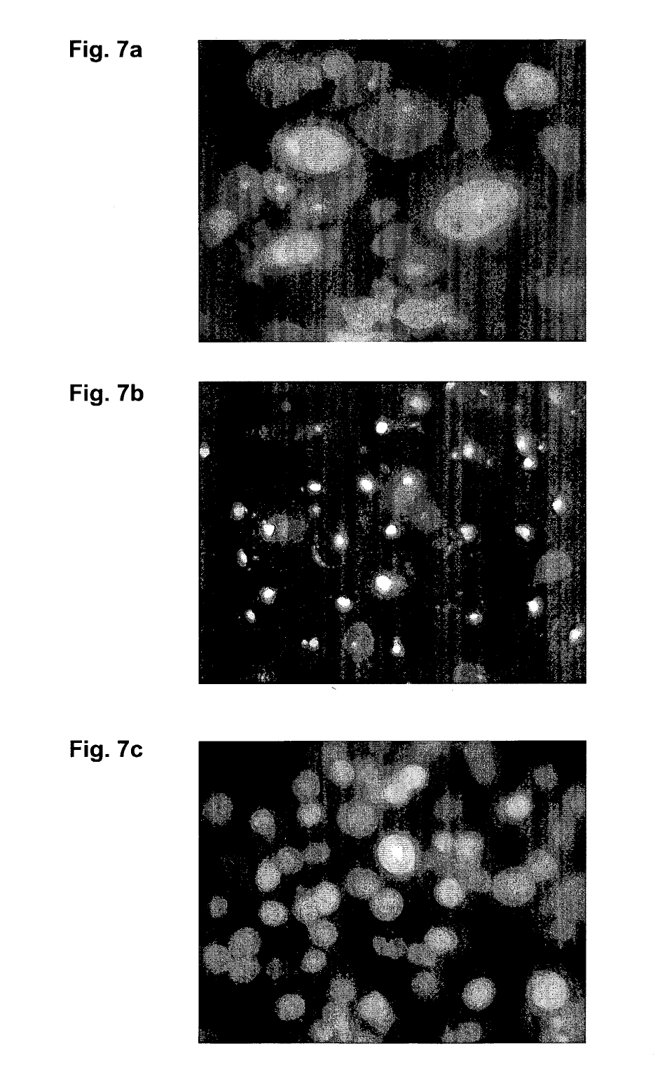

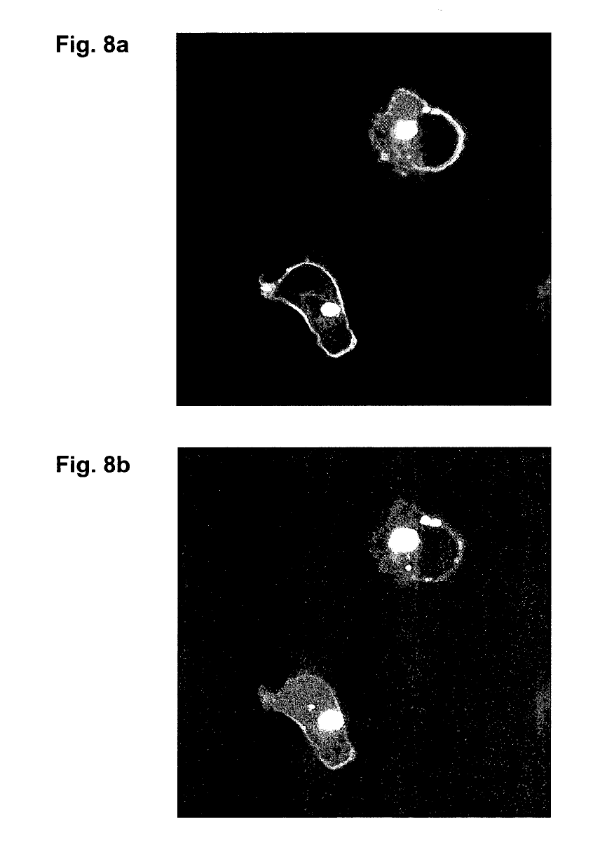

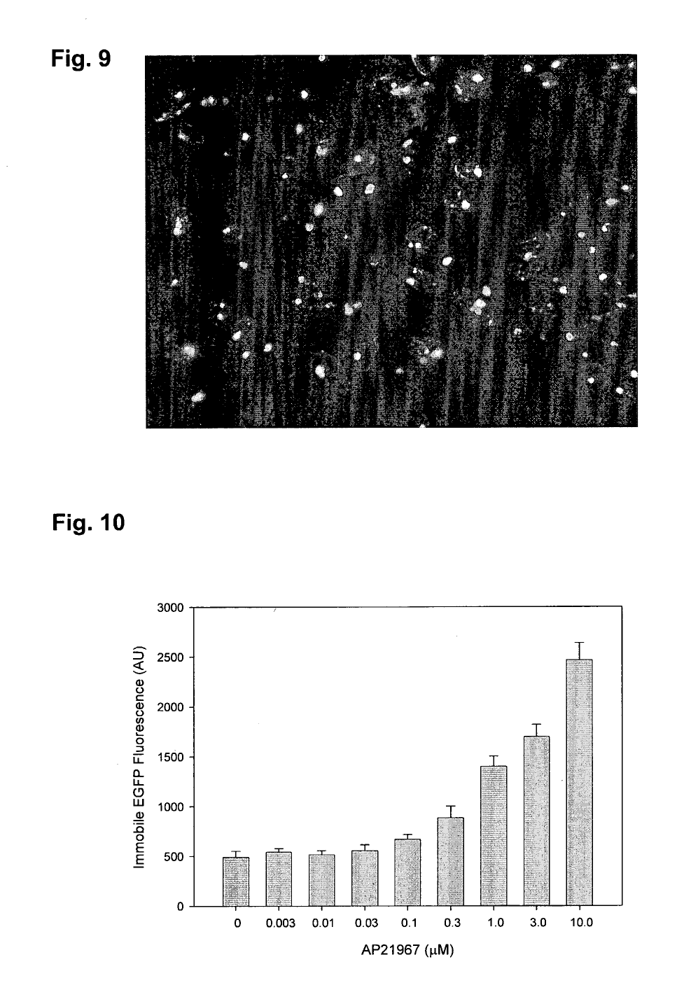

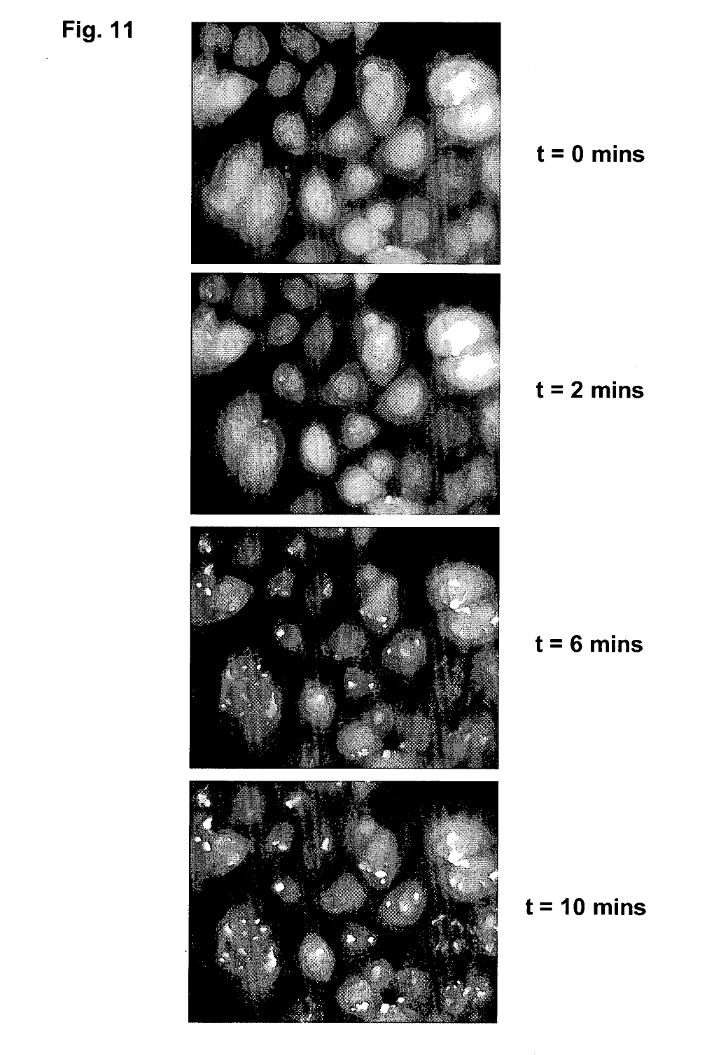

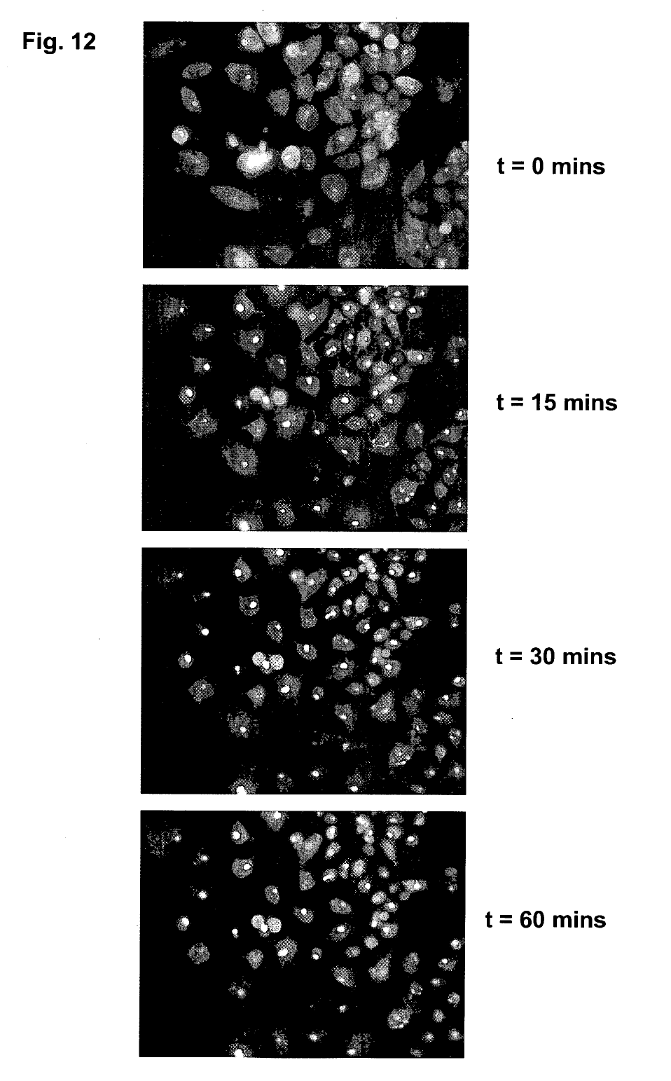

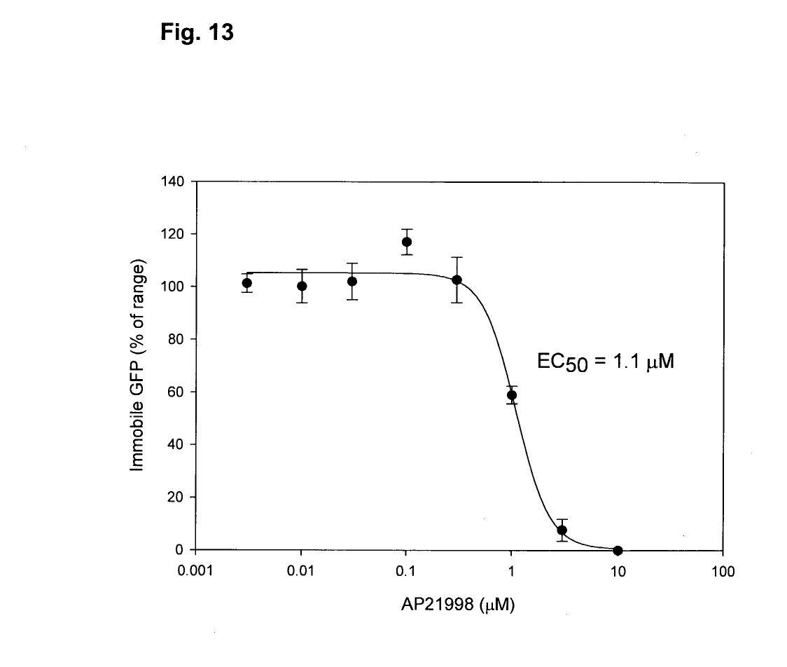

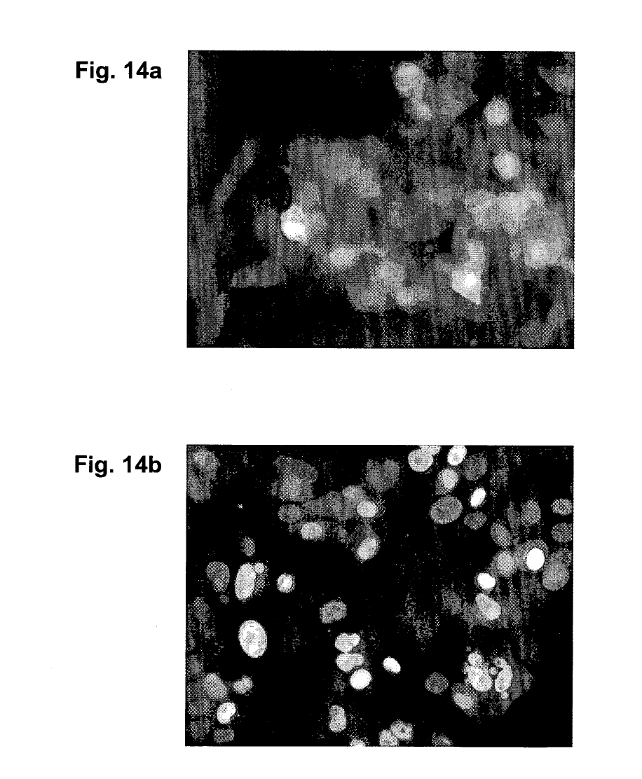

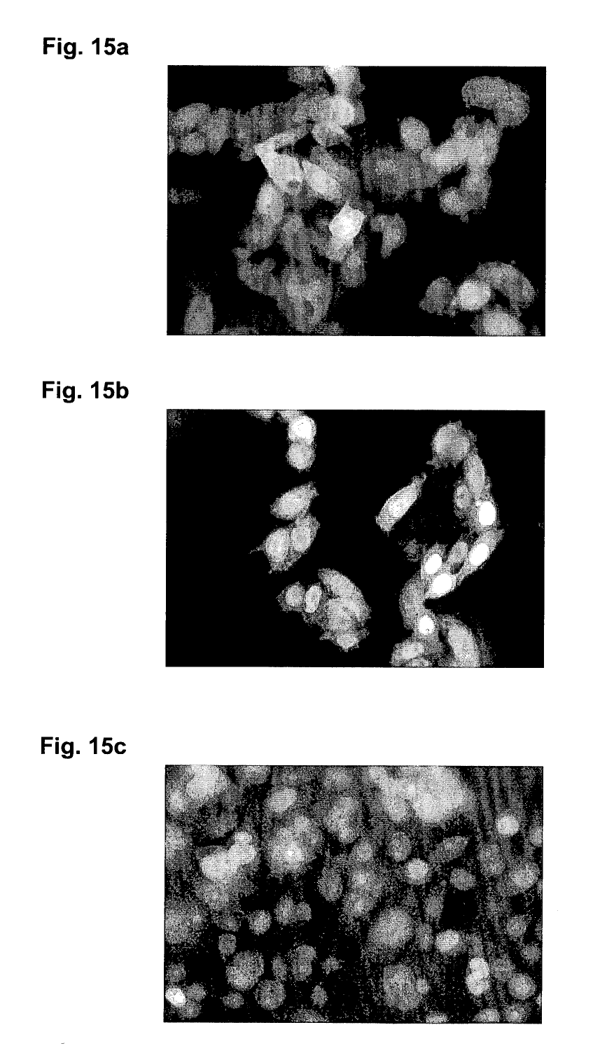

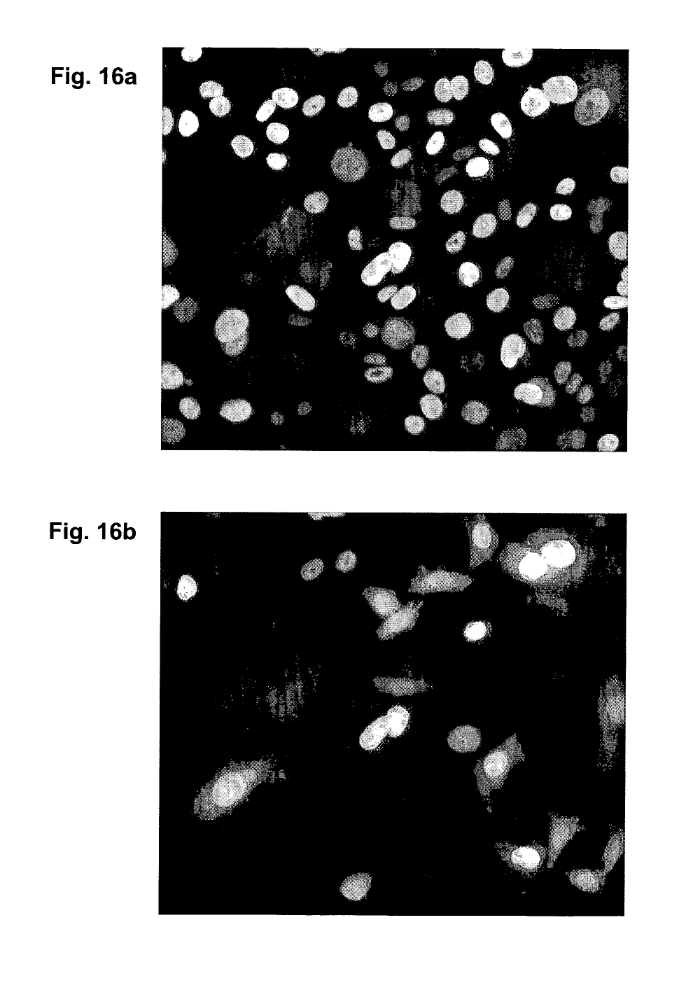

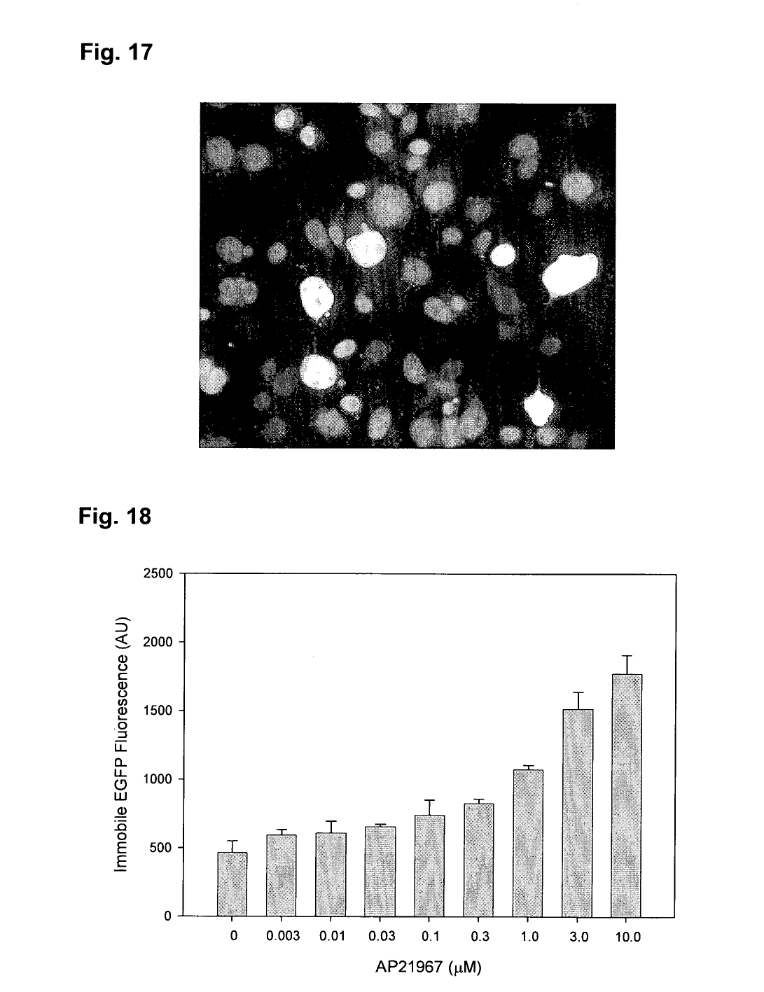

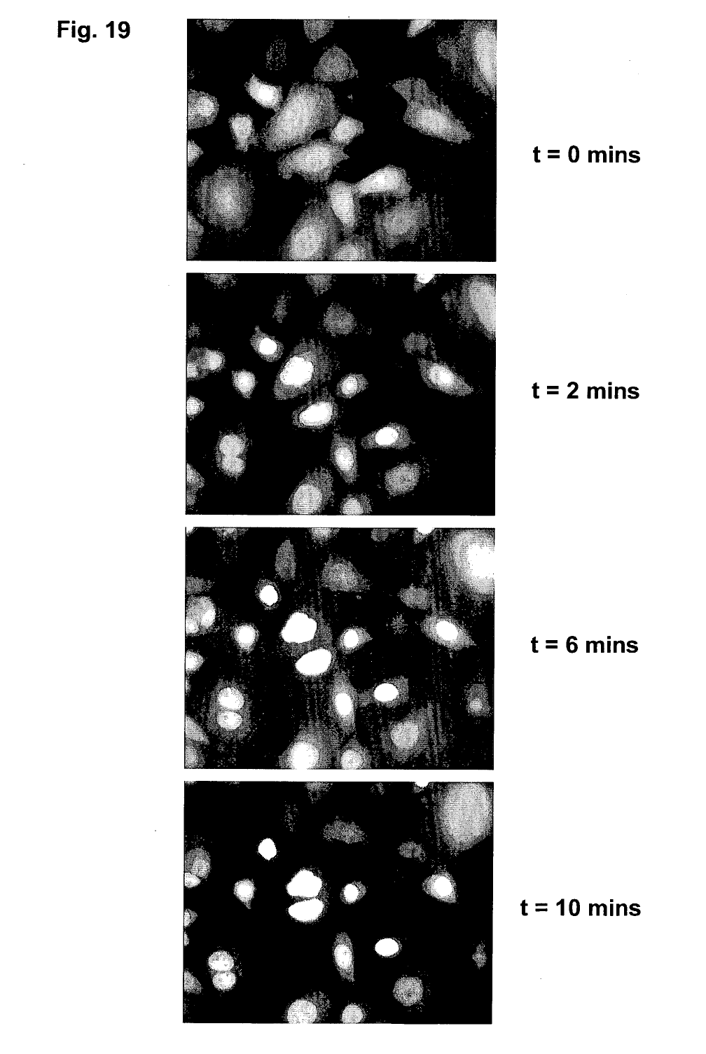

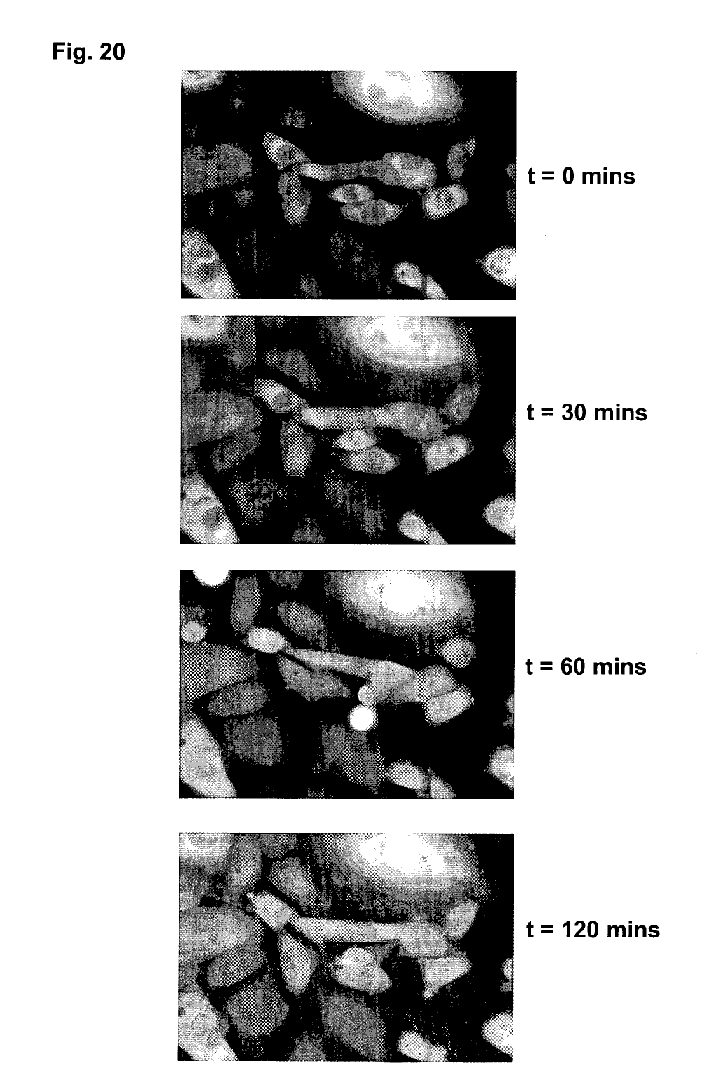

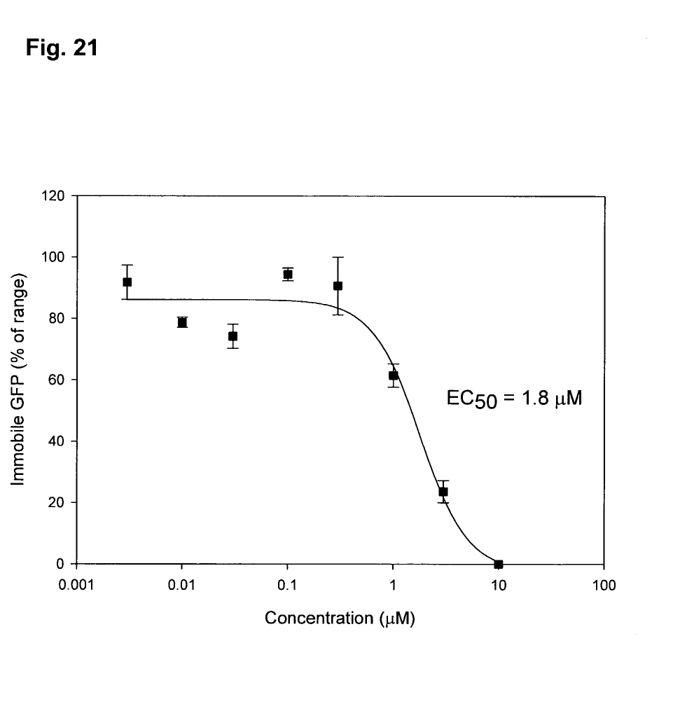

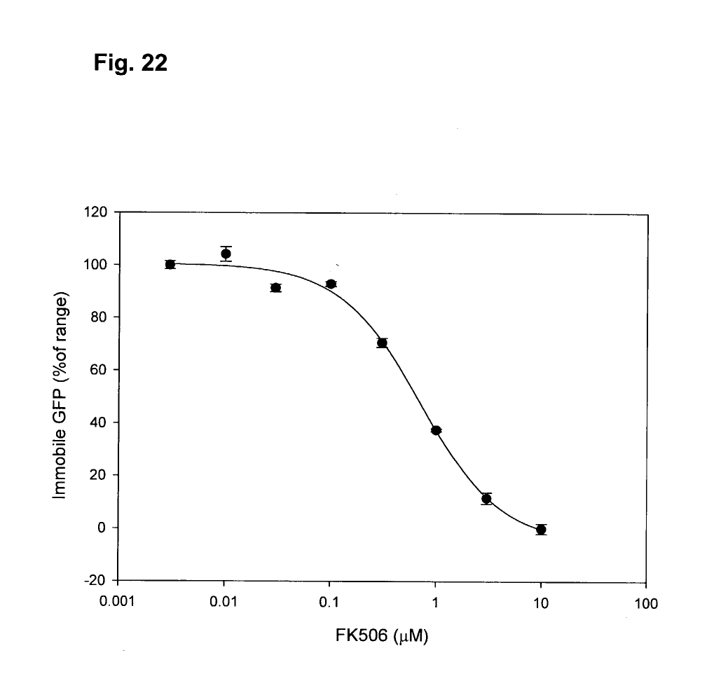

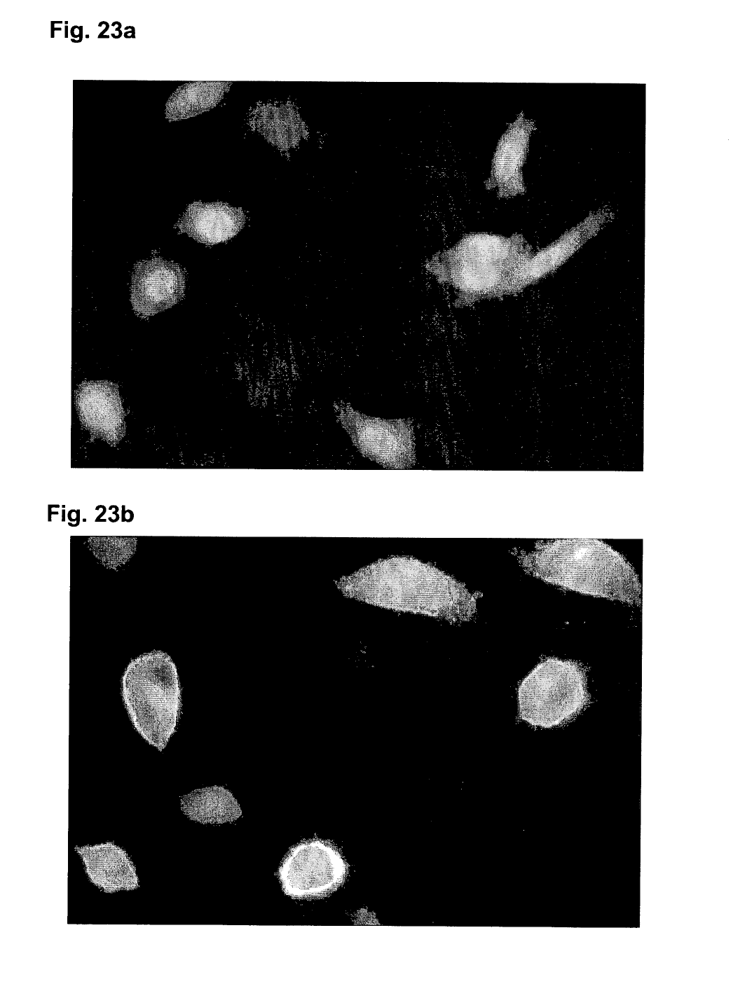

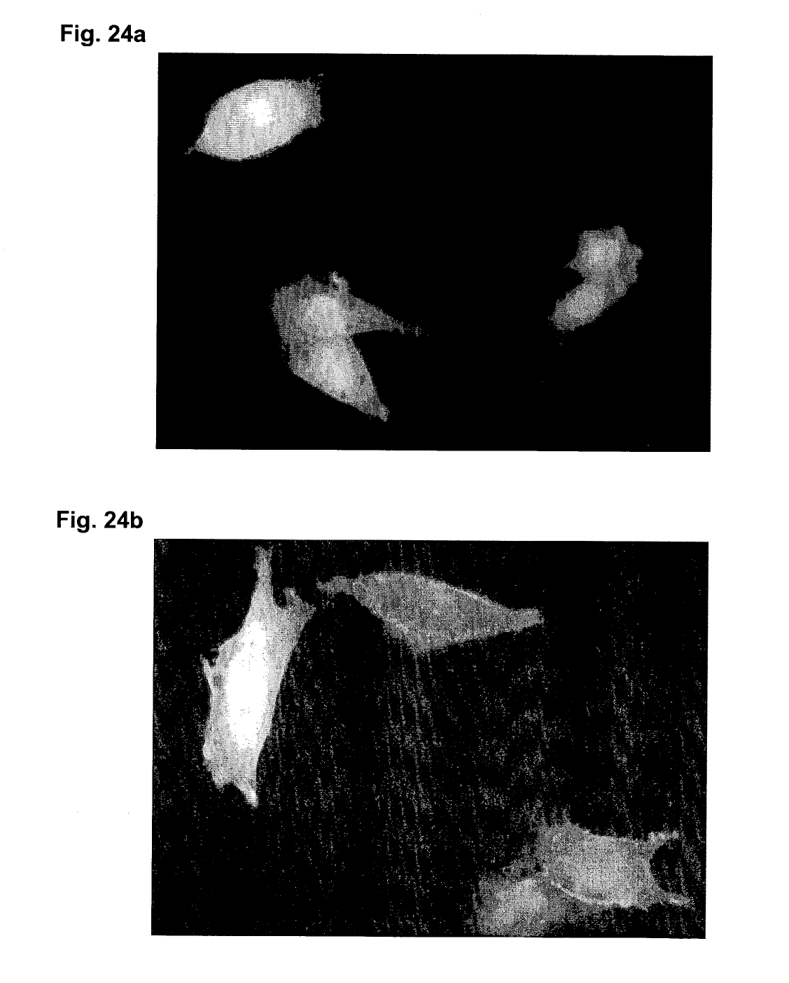

Schematic review of the iGRIP system for testing compounds:

A: The three conjugates are transfected into the cell in parallel or in sequence.

B: The optional interaction stimulus is applied. This will result in an interaction between the two proteins of interest.

C: The compound to test is added to the cells. If the compound is capable of breaking the interaction between the two freely floating proteins, it will do so.

D: The dimeriser compound is added. If the two proteins of interest are linked, the distribution of the detectable group will mimic the distribution of the anchor protein. If the two proteins of interest are not linked (e.g. due to an effect of the compound to be tested), the distribution of the detectable group will mimic the distribution of the second protein of interest.

FIG. 3:

Schematic review of the iGRIP system for testing interaction partners:

A: The anchor conjugate is transfected into the cell. The cells are selected on for stable expression and for suitable anchor localization.

B: The 2^{nd} conjugate is transfected into the cell. The cells are selected for reversible redistribution in response to the dimerizer compounds.

C: A library of 3^{rd} conjugates are transfected into the cells. All of these 3^{rd} conjugates comprises a protein of interest (typically an unknown protein) fused in frame to a detectable group.

D: The cells transfected with the 3^{rd} conjugate are selected for cells showing induced redistribution of the detectable group in response to applying the dimerizer compound. Such cells likely contain a fusion wherein the protein of interest binds to the first protein of interest.

FIG. 4a

Diagram showing the components of a 2-part iGRIP system, being an anchor conjugate comprising an anchor protein fused here to FRB*, and a detectable conjugate comprising here FKBP fused to EGFP.

FIG. 4b

Diagram showing linkage of the anchor and detectable conjugates by a dimerizer compound, here AP21967, being a compound able to link FRB* to FKBP specifically, and having no other biological activity in mammalian cells.

FIG. 5a

Diagram showing the components of a 3-part iGRIP system, being an anchor conjugate comprising an anchor protein fused here to FRB*, a mediator conjugate comprising FKBP fused to tandem CAD domains and a detectable conjugate comprising here tandem CAD domains fused to EGFP.

FIG. 5b

Diagram showing how the tandem CAD domains of the mediator and detectable conjugates spontaneously homodimerize.

FIG. 5c

Diagram showing linkage of the anchor and mediator+detectable complex by a dimerizer compound, here AP21967, being a compound able to link FRB* to FKBP specifically, and having no other biological activity in mammalian cells.

FIG. 5d

Diagram showing the effect of AP21998 on the 3-part system in the presence of dimerizer compound AP21967. Anchor and mediator conjugates remain attached, and only the detectable conjugate is stripped away by the CAD:CAD interaction inhibitor AP21998.

FIG. 6

Micrograph of CHO cells stably expressing the components of the 2-part ActinPaint system, imaged for EGFP. Cells in FIG. 6a are untreated, while those in FIG. 6b have been treated with 800 nM AP21967 for 60 minutes. EGFP fluorescence is recruited to stable cytoplasmic aggregates in the treated cells.

FIG. 7

Micrograph of CHO cells stably expressing the components of the 3-part ActinPaint system, imaged for EGFP. Cells in FIG. 7a are untreated, while those in FIG. 6b have been treated with 800 nM AP21967 for 60 minutes. EGFP fluorescence is recruited to stable cytoplasmic aggregates in the treated cells. Cells in FIG. 7c have been further treated with 5 μM of AP21998 for 2 hours in the continued presence of AP21967. The bright aggregates have dispersed into the cytoplasm.

FIG. 8

Confocal micrograph of CHO cells stably expressing the components of the 3-part ActinPaint system. The cells were first treated with 800 nM AP21967 for 60 minutes, then fixed and stained with rhodamine phalloidin (Molecular Probes Inc., Oregon, USA). FIG. 8a is the red channel image showing distribution of F-actin in the cells. FIG. 8b is the green channel image, showing the distribution of EGFP. It is apparent that the EGFP fluorescence colocalises with that of the phalloidin-labelled F-actin, demonstrating that the detectable conjugate has been recruited specifically to the F-actin structures by application of dimerizer compound.

FIG. 9

EGFP image of CHO cells stably expressing the components of the 3-part ActinPaint system. Cells were treated with 800 nM AP21967 for 60 minutes, then mobile EGFP-labelled components extracted following the procedure described in Examples 3 and 6. The immobile F-actin anchored fluorescence remains in the cells, and may be measured in a plate reader or by any of the other methods described in Example 3.

FIG. 10

Dose-response curve to AP21967 for CHO cells stably expressing the components of the 3-part ActinPaint system. The procedure for treatment, preparation and reading of the signal from the cells is detailed in Example 6. Results are shown corrected for background and cell number, each value being the mean±sd from 4 replicates. The response does not saturate in this experiment, but at 1000 nM AP21967 the immobile EGFP fluorescence has increased by 3-fold over background.

FIG. 11

Time series micrographs of CHO cells stably expressing the components of the 2-part ActinPaint system, treated with 800 nM AP21967 and thereafter imaged for EGFP at the times indicated. Recruitment of fluorescence to the cytoplasmic F-actin aggregates is visible after only 2 minutes, reaching a maximum response after approximately 10 minutes.

FIG. 12

Time series micrographs of CHO cells stably expressing the components of the 3-part ActinPaint system, treated with 800 nM AP21967 and thereafter imaged for EGFP at the times indicated. Recruitment of fluorescence to the cytoplasmic F-actin aggregates is visible after only a few minutes, reaching a maximum response after approximately 30-40 minutes.

FIG. 13

Dose-response curve to AP21998 for CHO cells stably expressing the components of the 3-part ActinPaint system and co-treated with 800 nM AP21967 dimerizer. The procedure for treatment, preparation and reading of the signal from the cells is detailed in Example 6. Results are shown corrected for background and cell number, each value being the mean±sd from 4 replicates. The EC₅₀ for AP21998 was approximately 1.1 μM for the prevention of recruitment of EGFP-labelled components to the F-actin aggregates.

FIG. 14

Micrograph of CHO cells stably expressing the components of the 2-part Histone H2B system, imaged for EGFP. Cells in FIG. 14a are untreated, while those in FIG. 14b have been treated with 800 nM AP21967 for 60 minutes. EGFP fluorescence is recruited to the nuclei in responding cells.

FIG. 15

Micrograph of CHO cells stably expressing the components of the 3-part Histone H2B system, imaged for EGFP. Cells in FIG. 15a are untreated, while those in FIG. 15b have been treated with 800 nM AP21967 for 60 minutes. EGFP fluorescence is recruited to the nuclei in responding cells.

Cells in FIG. 15c have been treated with 800 nM of AP21967+5 μM of AP21998 for 2 hours. There is no nuclear accumulation, over and above that seen in untreated cells, of EGFP-labelled components in the presence of AP21998.

FIG. 16

Confocal micrograph of CHO cells stably expressing the components of the 3-part Histone H2B system. The cells were first treated with 800 nM AP21967 for 60 minutes, then fixed and stained (in the red channel) for HA antigen as described in Example 7. FIG. 16a is the red channel image showing the exclusively nuclear distribution of HA-labelled Histone H2B anchor conjugate in the cells. FIG. 16b is the green channel image, showing the distribution of EGFP. It is apparent that the EGFP fluorescence colocalises with that HA-labelled Histone H2B anchor conjugate in responding cells.

FIG. 17

EGFP image of CHO cells stably expressing the components of the 3-part Histone H2B system. Cells were treated with 800 nM AP21967 for 60 minutes, then mobile EGFP-labelled components extracted following the procedure described in Examples 3 and 6. The immobile Histone-H2B anchored fluorescence remains in the cells, and may be measured in a plate reader or by any of the other methods described in Example 3.

FIG. 18

Dose-response curve to AP21967 for CHO cells stably expressing the components of the 3-part Histone H2B system. The procedure for treatment, preparation and reading of the signal from the cells is detailed in Example 7. Results are shown corrected for background and cell number, each value being the mean±sd from 4 replicates. The response does not saturate in this experiment, but at 1000 nM AP21967 the immobile EGFP fluorescence has increased by 2-fold over background.

FIG. 19

Time series micrographs of CHO cells stably expressing the components of the 2-part Histone H2B system, treated with 800 nM AP21967 and thereafter imaged for EGFP at the times indicated. Recruitment of fluorescence to the nuclei is visible after only 2 minutes, reaching a maximum response after approximately 10 minutes.

FIG. 20

Time series micrographs of CHO cells stably expressing the components of the 3-part Histone H2B system, treated with 800 nM AP21967 and thereafter imaged for EGFP at the times indicated. Recruitment of fluorescence to the nuclei is visible after approx 20 minutes, reaching a maximum response after approximately 60 minutes. The amount of cytoplasmic fluorescence remaining in these cells makes nuclear translocation less clear, but when treated for removal of mobile EGFP-labelled components (extraction procedure, Example 3), the translocation becomes easily measurable (see e.g. FIG. 15)

FIG. 21

Dose-response curve to AP21998 for CHO cells stably expressing the components of the 3-part Histone H2B system and co-treated with 800 nM AP21967 dimerizer. The procedure for treatment, preparation and reading of the signal from the cells is detailed in Example 7. Results are shown corrected for background and cell number, each value being the mean±sd from 4 replicates. The EC₅₀ for AP21998 was approximately 1.8 μM for the prevention of recruitment of EGFP-labelled components to the nuclear compartment.

FIG. 22

Dose-response curve to FK506 for CHO cells stably expressing the components of the 2-part Histone H2B system and co-treated with 800 nM AP21967 dimerizer. The procedure for treatment, preparation and reading of the signal from the cells is detailed in Example 7. Results are shown corrected for background, each value being the mean±sd from 4 replicates. The EC₅₀ for FK506 vs. 800 nM AP21967 was approximately 700 nM for the prevention of recruitment of the EGFP-labelled component to the nuclear compartment.

FIG. 23

Micrograph of CHO cells stably expressing the components of the 2-part Src(1-14) system, imaged for EGFP. Cells in FIG. 23a are untreated, while those in FIG. 23b have been treated with 500 nM AP21967 for 60 minutes. EGFP fluorescence is recruited to the plasma membrane in responding cells.

FIG. 24

Micrograph of CHO cells stably expressing the components of the 3-part Src(1-14) system, imaged for EGFP. Cells in FIG. 24a are untreated, while those in FIG. 24b have been treated with 500 nM AP21967 for 120 minutes. EGFP fluorescence is recruited to the plasma membrane in responding cells.

### EXAMPLES

### Example 1

Construction of the Probes and Fusions:

Below is described how specific plasmids encoding various fusions may be constructed. A person skilled in the art will realize that there are other ways to achieve similar results. For example, CAD may be constructed by introducing the F36M mutation by site specific mutagenesis in the coding sequence of FKBP. 2×CAD may be constructed by fusing two copies of CAD using PCR with partly overlapping primers. Similarly, FRB* may be constructed by introducing the T2098L mutation by site specific mutagenesis in the coding sequence of the FRAP. The relevant domain of FRAP may for example be isolated from a cDNA library as described above. The various fusions may be expressed from other vectors. When cells are transfected with more than one plasmid, selection markers of the plasmids should be different. Linker sequences between components of the fusions may differ depending on the exact nature of the construction. Linkers other than the ones described below may work well.

Construction of a First Plasmid Encoding a Plasma Membrane Anchored HA-Tagged FKBP Fusion Protein.

The coding sequence of human FKBP (GenBank Acc XM_{—}01660) is isolated from a cDNA library, e.g. fetus or heart or HeLa cDNA available from Clontech, using PCR and specific primers FKBP-top and FKBP-bottom described below. FKBP-top includes sequence from the N-terminal end of FKBP including the start codon of FKBP, and FKBP-bottom contains sequence from the C-terminal end of FKBP including the amino acid immediately preceding the stop codon.

The ca 0.33 kb PCR product is used as template in a second round of PCR with primers Scr-myr-top and HA-stop described below. Src-myr-top includes the following sequence elements: ACC immediately preceding an ATG start codon to provide an efficient Kozak sequence, the N-terminal 14 amino acids of c-src (GenBank Acc NM_{—}005417) that encode a myrisoylation signal to anchor the protein in the plasma membrane, and sequence specific to the N-terminal end of FKBP. HA-stop includes sequence encoding the antigenic peptide usually known as HA including a stop codon, and sequence specific to the C-terminal end of FKBP. Scr-myr-top and HA-stop also contain at their 5′-ends the unique sequence for a restriction enzyme to allow the ca. 0.4 kb PCR product to be ligated into an expression vector, e.g. as an EcoR1-BamH1 fragment into the expression vector pEF6/V5-His (Invitrogen). This places the c-src(1-14)-FKBP-HA fusion protein under the control of an EF-1alpha promoter on a plasmid containing blasticidin resistance as selectable marker in mammalian cells.

| | |
|---|---|
| FKBP-top: | 5′-ATgggAgTgCAggTggAAACC-3′ |
| FKBP-bottom: | 5′-TTCCAgTTTTAgAAgCTCCAC-3′ |
| Src-myr-top: | 5′-gTTgAATTCACCATgggTAgCAACAAgAgCAAgCCCAAggATgCCAgCCAgCggATgggAgTgCAggTggAAACC-3′ |
| HA-stop: | 5′-gTTggATCCTCAAgCgTAATCCggAACATCgTATgggTACATTTCCAgTTTTAgAAgCTCCAC-3′ |

Construction of a Second Plasmid Encoding a Myc-Tagged FRAP (FKBP Binding Domain)-SOS1 Fusion Protein.

The coding sequence of the FKBP binding domain of human FRAP (GenBank Acc XM_{—}001528, amino acids 2025-2114) is isolated from a cDNA library, e.g. fetus or heart or HeLa cDNA available from Clontech, using PCR and specific primers FRAP-top and FRAP-bottom described below. FRAP-top includes sequence from amino acid number 2025 of FRAP, and FRAP-bottom contains sequence from amino acid 2114 of FRAP, plus sequence specific to the N-terminal end of human SOS1 (GenBank Acc NM_{—}005633). The coding sequence human SOS1 is isolated from a cDNA library, e.g. fetus or brain cDNA available from Clontech, using PCR and specific primers SOS-top and SOS-stop described below. SOS-top includes sequence from the N-terminal end of SOS1 preceded by sequence from around amino acid 2114 of FRAP, and SOS-stop contains sequence from the C-terminal end of SOS1 followed by an Mlu1 restriction site.

The resulting ca 0.3 kb FRAP PCR product and 4 kb SOS1 PCR product are used next as templates together in a second round of PCR with primers Myc-top and SOS-stop described below. Myc-top includes the following sequence elements: ACC immediately preceding an ATG start codon to provide an efficient Kozak sequence, 13 amino acids encoding the antigenic sequence usually known as Myc-tag, and sequence specific to FRAP starting at amino acid 2025. At the 5′-ends, Myc-top also contains the unique sequence for a restriction enzyme to allow the ca 4.4 kb PCR product to be ligated into an expression vector, e.g. as a Xho1-Mlu1 fragment into the expression vector pZeoSV (Invision vector, e.g. as a Xho1-Mlu1 fragment into the expression vector pZeoSV (Invitrogen). This places the Myc-FRAP(2025-2114)-SOS1 fusion protein under the control of an SV40 promoter on a plasmid containing zeocin resistance as selectable marker in mammalian cells.

A T2098L mutation in FRAP is introduced by performing QuickChange mutagenesis (Stratagene) on the plasmid using PCR and primers FRAP-QC-top and FRAP-QC-bottom described below. This introduces the T2098L mutation and a diagnostic Pst1 restriction site.

| | |
|---|---|
| FRAP-top: | 5′-gAgATgTggCATgAAggCCTg-3′ |
| FRAP-bottom: | 5′-CTgCggCgCCTgCTgCATCTgCTTTgAgATTCgTCgg-3′ |
| SOS-top: | 5′-CgAATCTCAAAgCAgATgCAgCAggCgCCgCAgCCTTAC-3′ |
| SOS-stop: | 5′-gTTACgCgtTCATTggggAgTTTCTgCATTTTC-3′ |
| Myc-top: | 5′-gTTCTCgAgACCATggCATCAATgCAgAAgCTgATCTCAgAggAAgATCTTgAgATgTggCATgAAggCCTg-3′ |
| FRAP-QC-top: | 5′-gTCAAggACCTCCTgCAggCCTgggACCTC-3′ |
| FRAP-QC-bottom: | 5′-gAggTCCCAggCCTgCAggAggTCCTTgAC-3′ |

Construction of a Third Plasmid Encoding a Fusion Between GRB2 and a GFP, e.g. EGFP.

The coding sequence of human GRB2 (GenBank accession number NM_{—}002086) is isolated from e.g. a human fetus or brain or placenta cDNA library by PCR with primers 0073 and 0074 described below. The top primer includes specific GRB2 sequences following the ATG and a Hind3 cloning site. The bottom primer includes specific GRB2 sequence preceding the stop codon and an EcoR1 cloning site. The ca 0.65 kb PCR product is digested with restriction enzymes Hind3 and EcoR1, and ligated into pEGFP-N1 vector DNA (Clontech, Palo Alto, GenBank Accession number U55672) digested with Hind3 and EcoR1. This creates a fusion between GRB2 and EGFP under the control of a CMV promoter.

| | |
|---|---|
| 0073-top: | 5′-GCGAAGCTTTCAGAATGGAAGCCATCG-3′ |
| 0074-bottom: | 5′-GCCGAATTCGGACGTTCCGGTTCACG-3′ |

Construction of a cDNA Library to Express Random GFP Fusions in Mammalian Cells.

Construction of full-length cDNA libraries using current methodology requires that synthesis of the cDNA proceeds 3′ to 5′. That means that sequence containing the stop codon of any protein contained in the library will be present in the cDNA. Therefore, a library of protein fusions to GFP is constructed as GFP-proteinX fusions instead of proteinX-GFP fusions.

Construction of full-length cDNA libraries using current methodology cannot situate the 5′-end at the start codon. That means that sequence from the 5′-untranslated region will be part of the cDNA, and for statistical reasons, only one third of random protein fusions will be in frame with the upstream protein, in this case the GFP. To overcome this problem one may construct the libraries in such a way that all three reading frames are included. This will make the library representative of the members contained in it, but it will still contain a majority of irrelevant out-of-frame fusions.

Below is described an example of constructing a cDNA library to express random GFP fusions in mammalian cells, using commercially available components.

Several suppliers offer high quality cDNA libraries from a variety of tissues, e.g. Clontech Laboratories (Palo Alto, Calif.). Many of the libraries are prepared in such a way that the cDNA is flanked by a site for restriction enzyme Srf1 at the 5′-end and a site for restriction enzyme Not1 at the 3′-end. The recognition sites of these enzymes occur rarely in native DNA or cDNA, so therefore, the cDNA library will be mostly intact following digestion with these enzymes. Following digestion with Srf1 and Not1, the cDNA may now be inserted into a suitable vector in a directional manner. A suitable vector might be pEGFP-C1 (Clontech, GenBank Acc U55763) that encodes the EGFP derivative of GFP followed by a multiple cloning site. The MCS is first modified to accept Srf1-Not1 fragments, e.g. by converting the Bgl2 site to an Srf1 site using the following adaptor 5′-gATCgCCCgggC-3′ first, and next converting e.g. the Acc65 site (aka Kpn1) to a Not1 site using the following adaptor 5′-gTACgCggCCgc-3′. The cDNA library is cut with Srf1 and Not1 and ligated into the modified pEGFP-C1 vector. To construct a library with a fusion to GFP in all three reading frames, the vectors pEGFP-C2 and pEGFP-C3 from Clontech, that are similar to pEGFP-C1 but with the MCS shifted to the two alternative reading frames, are modified in the same fashion as pEGFP-C1 first, and next used as recipients of the Srf1-Not1 digested library along with the pEGFP-C1 derivative.

Construction of Plasmid PS1547.

Plasmid PS1547 encodes a fusion of EGFP and 2×CAD under the control of a CMV promoter and with kanamycin and G418 resistance as selectable marker in E.coli and mammalian cells, respectively.

Plasmid PS1547 was derived from plasmids pEGFP-C1 (Clontech) and pC4-FM-2E (Ariad).

pC4-FM-2E was digested with restriction enzymes Xba1 and Spe1, and the ca 0.65 kb fragment encoding 2×CAD was isolated, and ligated into the unique Xba1 site of vector pEGFP-C1, as Xba1 and Spe1 sites are compatible. The pEGFP-C1 DNA was prepared from a dam-minus E.coli strain as Xba1 is sensitive to overlapping dam methylation. A clone was isolated in which the orientation of the insert was 5′-Xba1/3′-Spe1-Xba1. This creates an in frame fusion between EGFP and 2×CAD, connected by a linker derived from vector sequence. This plasmid is called PS1547.

Construction of Plasmid PS1556.

Plasmid PS1556 encodes a fusion of FKBP and 2×CAD with a V5His6 tag under the control of an EF-1alpha promoter and with ampicillin and blasticidin resistance as selectable marker in E.coli and mammalian cells, respectively.

Plasmid PS1556 was derived from plasmids pC4-EN-F1 (Ariad) and plasmid PS1540. Plasmid PS1540 was derived from plasmids pC4-FM-2E (Ariad) and pEF6/V5-HisA (Invitrogen).

Construction of Intermediate PS1540.

pC4-FM-2E was digested with restriction enzymes Xba1 and Spe1, and the ca 0.65 kb fragment encoding 2×CAD was isolated, and ligated into the unique Xba1 site of vector pEF6/V5-HisA, as Xba1 and Spe1 sites are compatible. A clone was isolated in which the orientation of the insert was 5′-Xba1/3′-Spe1-Xba1. This creates an in frame fusion between 2×CAD and the V5Histag. This plasmid is called PS1540.

The coding sequence of FKBP was isolated from plasmid pC4-EN-F1 (Ariad) by PCR with primers 2197 and 2198 described below. The ca 0.32 kb fragment encoding FKBP was isolated, digested with restriction enzymes Acc65 and EcoR1, and ligated into PS1540 digested with with restriction enzymes Acc65 and EcoR1. This creates an in frame fusion between FKBP and 2×CADV5His, connected by a linker derived from vector sequence. This plasmid is called PS1556.

primers:

| | |
|---|---|
| 2197: | 5′-GTTGGTACCACCATGGGAGTGCAGGTGGAAACCATC-3′ |
| 2198: | 5′-GTTGAATTCTTCCAGTTTTAGAAGCTCCACATC-3′ |

Construction of Plasmid PS1208.

Plasmid PS1208 encodes a fusion of F64L,E222G-eGFP and FKBP12 under the control of a CMV promoter and with kanamycin and G418 resistance as selectable marker in E.coli and mammalian cells, respectively.

Plasmid PS1208 was derived from plasmid PS1040, which was derived from plasmid PS1000, which was derived from plasmid PS401, which was derived from plasmid pEGFP-C1 (Clontech).

Construction of Intermediate PS401.

pEGFP-C1, which contains the chromophore TYG, was modified to contain the wildtype chromophore SYG by PCR with primers 9859 and 9860 described below. These primers anneal to the plasmid around the chromophore, and produce a linear amplification product with the T65S (see note on numbering below) mutation, and a unique Spe1 site by silent mutation. The PCR product was digested with Spe1, and religated. This plasmid is called PS401.

Construction of Intermediate PS1000.

PS401 was subjected to QuickChange mutagenesis with primers 0226 and 0225 described below. This introduces the E222G mutation (see note on numbering below) and a diagnostic Avr2 site by silent mutation. This plasmid is called PS1000.

Construction of Intermediate PS1040.

PS1000 was digested with restriction enzymes Xho1 and BamH1, which cut in the multiple cloning site 3′ of the GFP, blunt-ended with Klenow, and ligated with Gateway reading frameA cassette (from Invitrogen). A plasmid was isolated in which a single copy of reading frameA was inserted with its 5′-end ligated to the blunt-ended Xho1 site, and the 3′-end of reading frameA ligated to the blunt-ended BamH1 site. This creates a Gateway compatible destination vector which accepts inserts in frame with the GFP. This construct is called PS1040.

Construction of Plasmid PS1208.

The coding sequence of FKBP12 (GenBank Acc XM_{—}016660) was isolated from human cDNA by PCR with primers 1271 and 1272 described below. The ca 0.35 kb product was first transferred into Gateway donor vector pDONR207 (Invitrogen) and then into Gateway destination vector PS1040. This creates an in frame fusion between the F64L,E222G-eGFP and FKBP, connected by a linker derived from vector sequence.

note on numbering: All GFPs derived from EGFP from Clontech contain an extra amino acid at position two to provide an optimal translation initiation sequence. This extra amino acid is not taken into account when referring to mutations in GFP, the numbering is relative to wildtype GFP.

primers:
9859:
5′-tgtactagtgaccaccctgtcttacggcgtgca-3′
9860:
5′-ctgactagtgtgggccagggcacgggcagc-3′
0226:
5′-cgcgatcacatggtcctcctagggttcgtgaccgccgccggg-3′
0225:
5′-cccggcggcggtcacgaaccctaggaggaccatgtgatcgcg-3′
1271:
5′-attB1-ccatgggagtgcaggtggaaacc-3′
1272:
5′-attB2-gtcattccagttttagaagctc-3′

Construction of Plasmid PS1570.

Plasmid PS1570 encodes a fusion of the actin binding domain of alpha-actinin (amino acids 1-133, named ActinPaint) and a modified version of the FKBP binding domain of FRAP (T2098L, named FRB*) with an HA tag under the control of a CMV promoter and with zeocin resistance as selectable marker in E.coli and mammalian cells.

Plasmid PS1570 was derived from plasmids PS275 and plasmid PS1549. Plasmid PS1549 was derived from plasmids PS1430 and PS1534. Plasmid PS1534 was derived from plasmids PS609 and pC4-RHE (Ariad). Plasmid PS609 was derived from plasmids pEGFP-C1 (Clontech) and pZeoSV (Invitrogen).

Construction of Intermediate PS609

The kanamycin/neomycin resistance marker on pEGFP-C1 was replaced with a zeocin resistance marker by digesting pEGFP-C1 with Avr2, which excises neomycin, and ligating the vector fragment with a ca 0.5 kb Avr2 fragment encoding zeocin resistance. This fragment was isolated by PCR using primers 9655 and 9658 described below with pZeoSV (Invitrogen) as template. Both primers contain Avr2 cloning sites, and flank the zeocin resistance gene including its E.coli promoter. The top primer 9658 spans the Ase1 site at the beginning of zeocin, which can be used to determine the orientation of the Avr2 insert relative to the SV40 promoter which drives resistance in mammalian cells. The resulting plasmid is referred to as PS609.

Construction of Intermediate PS1534.

Plasmid pC4-RHE (Ariad) was digested with restriction enzyme Xba1, blunt-ended with Klenow, and digested with BamH1. This excises the ca 0.3 kb FRB*-HA sequence from the plasmid. The fragment was ligated into PS609 digested with EcoR1, blunt-ended with Klenow, and digested with BamH1. This produces a fusion between EGFP and FRB*-HA. Both EcoR1 and Xba1 sites were restored by ligation of the blunt ends. This plasmid is called PS1534.

Construction of Intermediate PS1430.

A zeocin resistant derivative of pEGFP-C1 (Clontech) was digested with restriction enzymes Age1 and Bgl2. This excises EGFP from the plasmid. The vector fragment was ligated with annealed oligos 1478 and 1479 described below. This replaces EGFP with c-src(1-14). This plasmid is called PS1430.

Construction of Intermediate PS1549.

Plasmid PS1430 was digested with restriction enzymes SnaB1 and Bgl2, and the ca 0.35 kb fragment was ligated into the vector fragment of plasmid PS1534 digested with SnaB1 and Bgl2. This replaces EGFP with c-src(1-14) and creates a C-src(1-14)-FRB*-HA fusion connected by a linker derived from vector sequence. This plasmid is called PS1549.

Construction of PS1570.

The N-terminal 133 amino acids of alpha-actinin (Gen Bank Acc X15804), which comprise an actin binding domain, was isolated from a human placenta cDNA library (from Clontech) by PCR with primers 9656 and 9657 described below. The ca. 0.4 kb product was digested with restriction enzymes Hind3 and BamH1, cloned into pEGFP-N1 (Clontech) digested with Hind3 and BamH1. This construct is called PS275. The actin-binding domain of alpha-actinin was reisolated from PS275 by PCR with primers 2201 and 2202 described below. The ca 0.4 kb product was digested with restriction enzymes Acc65 and BamH1, and ligated into plasmid vector PS1549 digested with Acc65 and Bgl2. This replaces C-src(1-14) with the N-terminal 133 amino acids of alpha-actinin (called ActinPaint) and creates an ActinPaint-FRB*-HA fusion connected by a linker derived from vector sequence.

| primers: | |
|---|---|
| 9658-top: | TCCTAGGCTGCAGCACGTGTTGACAATTAATCATCGG-3′ |
| 9655-bottom: | TCCTAGGTCAGTCCTGCTCCTCGGCCACGAAGTGCAC-3′ |
| 1478: | 5′-ccggtaccatgggatccaacaagagcaagcccaaggatgccagccagcgga-3′ |
| 1479: | 5′-gatctccgctggctggcatccttgggcttgctcttgttggatcccatggta-3′ |
| 9656: | 5′-cctcctaagcttatcatggaccattatgattc-3′ |
| 9657: | 5′-cctcctggatccctgcgcaggatgatggtccag |
| 2201: | 5′- GTTGGTACCACCATGGACCATTATGATTCTCAG-3′ |
| 2202: | 5′- GTTGGATCCGCGCAGGATGATGGTCCAGATCATGCC |

Construction of Plasmid PS1569.

Plasmid PS1569 encodes a fusion of histone H2B and a modified version of the FKBP binding domain of FRAP (T2098L, named FRB*) with an HA tag under the control of a CMV promoter and with zeocin resistance as selectable marker in E.coli and mammalian cells.

Plasmid PS1569 was derived from plasmid PS1549 described above.

The coding sequence of H2B (GenBank Acc no NM_{—}003518.2) was isolated from a human cDNA library by PCR with primers 2205 and 2206 described below. The ca 0.4 kb product was digested with restriction enzymes Acc65 and Bgl2, and ligated into plasmid vector PS1549 digested with Acc65 and Bgl2. This replaces C-src(1-14) with H2B and creates an H2B-FRB*-HA fusion connected by a linker derived from vector sequence.

2205:
5′-GTTGGTACCACCATGCCAGAGCCAGCGAAGTCTGCTCCC-3′
2206:
5′-GTTAGATCTCTTAGCGCTGGTGTACTTGGTGACGGC-3′

| | | Nucleotide | Protein |
|---|---|---|---|
| Plasmid | Description | SEQ ID NO: | SEQ ID NO: |
| 1547 | EGFP-2xCAD | 1 | 2 |
| 1556 | FKBP-2xCAD-V5His | 3 | 4 |
| 1208 | F64L,E222G-eGFP-FKBP | 5 | 6 |
| 1570 | ActinPaint-FRB*-HA | 7 | 8 |
| 1569 | H2B-FRB*-HA | 9 | 10 |
| 1549 | c-src(1-14)-FRB*-HA | 11 | 12 |

### Example 2

Transfection and Cell Culture:

This example describes protocols and methods used for in vivo expression of the probes described in Example 1, and the visualization and measurement of changes undergone by EGFP fusion probes, either transfected singly or as co-transfections with anchor probes and in some cases mediator probes in CHO cells.

Chinese hamster ovary cells (CHO), are transfected with the plasmids described in Example 1 above, either using a single species of plasmid, or several plasmids co-transfected simultaneously, using the transfection agent FuGENE™ 6 (Boehringer Mannheim Corp, USA) according to the method recommended by the suppliers. Stable trans-fectants of single anchor-FKBP probes are selected using the appropriate selection agent, usually 5 μg/ml blasticidin HCl (Calbiochem) in the growth medium (HAM's F12 nutrient mix with Glutamax-1, 10% foetal bovine serum (FBS), 100 μg penicillin-streptomycin mixture ml⁻¹ (GibcoBRL, supplied by Life Technologies, Denmark)). Co-transfected cells are cultured in the same medium, but with the addition of two or three selection agents appropriate to the plasmids being used, usually 5 μg/ml blasticidin HCl plus 1 mg/ml zeocin and/or 0.5 mg/mlG418 sulphate. Cell are cultured at 37° C. in 100% humidity and conditions of normal atmospheric gases supplemented with 5% CO₂.

Clonal cell lines with particular properties are sub cultured from mixed populations of stably transfected cells by isolating individual cells and removing them to sterile culture flasks containing fresh culture medium with 5 μg/ml blasticidin HCl or 0.5 mg/ml G418 sulphate+1 mg/ml zeocin and/or 0.5 mg/ml G418 sulphateas appropriate to the plasmid(s) being selected.

### Example 3

Imaging

Automated Imaging

For fluorescence microscopy, cells are allowed to adhere to Lab-Tek chambered cover glasses (Nalge Nunc International, Naperville USA) for at least 24 hours and are then cultured to about 80% confluence. Cells can also be grown in plastic 96-well plates (Polyfiltronics Packard 96-View Plate or Costar Black Plate, clear bottom; both types tissue culture treated) for imaging purposes. Prior to experiments, the cells are cultured over night without selection agent(s) in HAM F-12 medium with glutamax, 100 μg penicillin-streptomycin mixture ml⁻¹ and 10% FBS. This medium has low auto fluorescence enabling fluorescence microscopy of cells straight from the incubator. For certain tests requiring medium of defined composition, particularly with regard to the presence of specific cell growth factors, the HAM's culture medium is replaced prior to imaging with a buffered saline solution (KRW buffer) containing (in mM) 3.6 KCl, 140 NaCl, 2 NaHCO₃, 0.5 NaH₂PO₄, 0.5 MgSO₄, 1.5 CaCl₂, 10 Hepes, 5 glucose, pH7.4.

An example of an automated imaging procedure is given in Appendix A, application number PCT/DK01/00466, filed Mar. 07, 2001 (filed together with the present application) examples 2 and 3.

Confocal Imaging:

Confocal images are collected using a Zeiss LSM 410 microscope (Carl Zeiss, Jena, Germany) equipped with an argon ion laser emitting excitation light at 488 nm. In the light path are a FT510 dichroic beam splitter and a 515 nm long-pass filter or a 510 to 525 nm band pass emission filter. Images are typically collected with a Fluar 40X, NA: 1.3 oil immersion objective, the microscope's confocal aperture set to a value of 10 units (optimum for this lens).

Time Lapse Sequences and Analysis:

Image sequences of live cells over time (time lapse) are gathered using a Zeiss Axiovert 135M fluorescence microscope fitted with a Fluar 40X, NA: 1.3 oil immersion objective and coupled to a Photometrics CH250 charged coupled device (CCD) camera (Photometrics, Tucson, Ariz. USA). The cells are illuminated with a 100 W HBO arc lamp. In the light path are a 470±20 nm excitation filter, a 510 nm dichroic mirror and a 515±15 nm emission filter for minimal image background. The cells are maintained at 37° C. with a custom-built stage heater.

Time lapse response profiles are extracted from image sequences using a region of interest (ROI) defined over the same co-ordinates or pixels for each successive image in a sequence: pixel values are summed and averaged over the ROI in each image, and the resulting values plotted against image number to generate a time lapse response profile for that defined region of the sequence. A ROI can include many cells, a single cell, or a region within a single cell.

Extraction Procedure:

The extraction procedure comprising simultaneous fixation+permeabilization, is useful to remove non-localized (i.e. mobile) GFP probe from the cytoplasm. This procedure involves a single fixation process incorporating 0.4% to 2% formaldehyde buffer (10% to 50% strength Lillies fixative) plus 0.2% to 1% Triton X-100. The actual concentrations used need to be optimized for the cell type being used; for typical CHO cells 2% formaldehyde+1% Triton X-100 gives excellent results. The combined fixative+detergent are applied to the cells for 10 to 20 minutes at room temperature. Cells are then washed three times with phosphate buffered saline. Nuclear DNA is stained with 10 μM Hoechst 33258 (Molecular Probes, Eugene, Oreg., USA) in PBS for 10 minutes at 25° C., then washed twice in PBS. Automated images are collected on a Nikon Diaphot 300 (Nikon, Japan) using a Nikon Plan Fluor 20X/0.5NA objective lens. The basic microscope is filted with a motorized specimen stage and motorized focus control (Prior Scientific, Fulbourn, Cambridge UK), excitation filter wheel (Sutter Instruments, Novato Calif. USA) and Photometrics PXL series camera with a KAF1400 CCD chip (Photometrics, Tucson, Ariz. USA), each of these items being under the control of a Macintosh 7200/90 computer (Apple Computer, Cupertino, Calif. USA). Automation of stage positioning, focus, excitation filter selection, and image acquisition is performed using macros written in-house, running under IPLab Spectrum for Macintosh (Scanalytics, Fairfax, Va. USA). Fluorescence illumination comes from a 100 W HBO lamp.

Images are collected in pairs, the first using a 340/10 nm excitation filter, the second with a 475RDF40 excitation filter (Chroma, Brattleboro, Vt.). Both images are collected via the same dichroic and emission filters, that are optimized for EGFP applications (XF100 filter set, Omega Optical, Brattleboro, Vt.). While the choice of filters for imaging the nuclear stain (Hoechst 33258) is not well matched to that dye's spectral properties, resulting in lower image intensity, it greatly improves the throughput of the procedure by allowing both images to be collected using the same dichroic and emission filter. This eliminates any image registration problems and focus shifts that would result from using two different filter sets, that would require more steps in the acquisition procedure and more extensive image processing to overcome.

The necessary images are collected as follows: A holder containing four 8-well cover glass chambers, or a single 96-well plate, is loaded onto the microscope. The program is started, and the first well of cells is moved into position and manually coarse-focused by the operator. The image is fine-focused by an auto-focus routine using the 340/10 excitation. An image is captured and stored at this excitation wavelength (the nuclear image), and then a second image is captured and stored at the longer wavelength excitation (the GFP image). The stage is automatically repositioned and microscope automatically refocused to capture a second pair of images within the same well. This process is repeated a set number of times (typically 4 to 8) for the first well. The stage then advances the next well to the imaging position, and the process repeats itself until the set number of image pairs has been captured from each well of cells.

The use of the combined fixation+permeabilization method greatly improves the signal over background for measurements of immobile versus mobile fluorophore accumulations in cells, particularly when the measurements are made on fluorescent plate readers.

This procedure is described in detail in WO 02/03072, on page 16, line 31 to page 20 line 25 and examples 2, 10, 11, 12, 13, and 14.

FLIPR™ Measurement

Redistributions of fluorescent probes from cytoplasm to plasma membrane may be quantified by standard imaging methods using simple image analysis of the changes in fluorescence intensity of cytoplasmic ROIs. Similar redistributions may also be measured on the FLIPR, and even on standard fluorescent plate readers, especially those configured to measure signal from adherent cells in micro titer plates.

Use of the FLIPR to measure PKC-like redistributions (of which EGFP-Cys1(PKCγ) is an example), and also PKAc-like redistributions, in real time, is detailed in an earlier patent AN IMPROVED METHOD FOR EXTRACTING QUANTITATIVE INFORMATION RELATING TO AN INFLUENCE ON A CELLULAR RESPONSE (WO 00/23615). FIG. 7 shows how the effect of a compound on the redistribution of Cys1γ-EGFP to the plasma membrane can be quantified using the FLIPR.

To optimize the use of the membrane translocation an enhancer compound is added to the cell/cell medium. Such addition will enhance the signal component of the redistribution response while only causing a marginal increase in assay background and cell-free plate background.

One such compound is Trypan Blue (CAS No. 72-57-1). Despite the fact that Trypan Blue is outside the cells, it reduces the fluorescence from GFP-tagged protein aggregated at the inner face of the plasma membrane resulting in an enhanced signal change as the protein redistributes from the cytosol to the membrane (decrease in signal) or from the membrane to the cytosol (increase in signal). Trypan Blue works well at 200 μM.

Another such compound is Acid Red 88 (CAS No. 1658-56-6). Acid Red 88 is water soluble but more lipophilic than Trypan Blue, and probably enters the cells to some extent and in a concentration-dependent manner. Thus, Acid Red 88 enhances the signal component at concentrations of about 5 μM. This and other anchor components are described in WO 01/81917.

### Example 4

Probes for SOS1 and GRB2 Interactions with c-SRC Anchor and a FRB-FKBP Mediator System

The present example describes generic ways to produce a cell line suitable for screening compounds targeting against a specific interaction between two partner components X and Y. It also describes briefly how such a cell line could be screened with a library of compounds. In these examples, the cells are derived from CHO cells co-transfected with three plasmids, one coding for fusion probes with interactor A or B attached to either the C or N terminus of the anchor moiety (the anchor probe), the second with the other interactor (A or B as appropriate) attached to bait or prey (in two possible orientations, the mediator probe) and the third with either bait or prey (as appropriate) attached to either the C or N terminus of GFP (the detectable probe). Anchor and detectable probes use different selection markers to ensure that cells under selection maintain all three plasmids; for example, the anchor may confer resistance to blasticidin, the detectable to neomycin and the mediator to zeocin. Cells that maintain all three probes under continuous selection (minimum of 2 weeks) are termed “stable”.

In this example, the anchor probes are based on the first 14 amino acids of the human c-SRC protein, that through myristoylation successfully directs itself to the plasma membrane. The membrane localization of the anchor can be detected with an antibody directed against the HA-tag included in the anchor fusion protein. Furthermore, in this example the anchor-mediator interaction is based on the inducible/reversible binding of FRB(T2098L) to FKBP12. Addition of heterodimerizer compound such as 100 nM of AP21967 (ARIAD Pharmaceuticals) or 100 nM of Rapamycin (SIGMA-Aldrich) leads to recruitment of the mediator protein fusion to the plasma membrane within minutes through induced heterodimerization of the FRB(T2098L) and FKBP12 moieties. The induced membrane localization of the mediator protein can be monitored by an antibody directed against the myc tag included in this fusion protein. As SOS1 and GRB2 interact in a cytosolic environment, addition of heterodimerizer compound redistributes GFP fluorescence to the plasma membrane. Subsequent removal of heterodimerizer compound or addition of competitor compound such as FK506 and derivatives (ARIAD Pharmaceuticals) will lead to reversal of the fluorescence distribution to the pattern observed before the addition of heterodimerizer compound meaning that FK506 can be used as a general reference compound for screening purposes. The following protocol is applied

1) Transfect a c-SRC anchor into CHO and generate a stable cell line by selecting with 5 μg/ml blasticidin HCl.

5 2) Check for membrane localization using HA antibody. If all cells show similar expression levels and robust membrane localization, proceed to 3). Otherwise, clone cells by dilution and check individual clones for membrane localization. Re-iterate until a homogenous cell line with robust membrane localization of the anchor has been obtained. This anchor cell line can be used as a starting point in future experiments and, thus, only needs to be generated once for each anchor type.

3) Co-transfect mediator and detectable probe pair into anchor cell line and select with 5 μg/ml blasticidin HCl, 1 mg/ml zeocin, and 0.5 mg/ml G418 sulphate.

4) Test cell line without and with 100 nM of AP21967 for 2 hrs.

a. Robust membrane redistribution of GFP in the presence of AP21967: clone cells until cell line is homogenous. At this point, the cell line is ready for screening.

b. no membrane localization of GFP in the presence of AP21967: go to 5)

c. Membrane localization of GFP in the absence of AP21967: choose another combination of anchor/mediator/detectable probes and start again.

5) Stain for localization of mediator with myc antibody.

a. Mediator is membrane-localized: test for conditional association of X and Y (see A below).

b. Mediator is not membrane-localized: anchor system not present or anchor/mediator pair not working—repeat or try another anchor/mediator system or orientation.

6) If no association conditions are found, choose another combination of anchor/mediator/detectable probes and start again.

Test for conditional association with an appropriate interaction stimulus:

A. i) incubate stables for 2-24 hrs with 100 nM AP21967

ii) test with an interaction stimulus, i.e. a treatment likely to bring the pair together. NB this may need time-lapse to catch transients. If robust fluorescence is observed at the plasma membrane, go to 4a.). If not, go to 6).

The second part of this example details how a specific cell line obtained using the procedure described above can be screened with a library of compounds in order to find inhibitors of the interaction between X and Y. In this specific example X and Y are chosen as the GRB2 and Sos proteins and compounds are sought that inhibit the specific interaction between GRB2 and Sos. So the cell line will contain the following three protein fusions Src(1-14)-FKBP12-Myc, HA- FRB(T2098L)-GRB2, and Sos-GFP, and GFP is detectable at the plasma membrane only in the presence of AP21967.

Protocol for c-SRC Anchor/FRB-FKBP Mediator System:

1) Seed cells at the required density (typically 50000 cells per well, containing 300 μl of culture medium) in 96 well plates. Other well formats are useful alternatives with the cell number per well adjusted accordingly to the volume of medium contained in the well.

2) Incubate overnight under proper growth conditions as outlined in Example 2.

3) Add compounds from compound library to cells at working concentration (typically 12 μM). Add compounds so that each well contains only one compound and so that a few wells in each plate are used for controls—the controls being no compound (vehicle only) for minimum response, and a competitive dose of FK506 (typically 1 μM) to give the maximum response possible. Incubate for 30 minutes.

4) Induce dimerization of FKBP12 and FRB(T2098L) with 100 nM AP21967 and follow redistribution of GFP to the plasma membrane as outlines in Example 3 above.

5) Identify hit compounds as those that significantly alter the redistribution of GFP to the plasma membrane, the significant dynamic range of the screening assay being defined by the controls listed under 3.

### Example 5

Probes for the Use of c-SRC Anchor/FRB-FKBP Dimerizer System to Screen for Novel Interactors of Protein X

This example describes generic ways to produce a cell line suitable for screening for novel proteinaceous interactors of protein X. In the present example, the cells are derived from CHO cells co-transfected with two plasmids, one coding for fusion probes with interactor A or B attached to either the C or N terminus of the anchor moiety (the anchor probe), the second with the other interactor (A or B as appropriate) attached to the bait molecule X (in two possible orientations, the bait probe). As outlined above, anchor and detectable probes use different selection markers to ensure that cells under selection maintain both plasmids. Cells that maintain both probes under continuous selection (minimum of 2 weeks) are termed “stable”.

In this example, the anchor probes are identical to those described in the previous examples. Furthermore, the anchor probe-bait probe interaction is identical to the anchor-mediator interaction described in Example 7. The induced membrane localization of the bait probe can be monitored by an antibody directed against the myc tag included in this fusion protein.

Protocol for c-SRC Anchor/FRB-FKBP Dimerizer System:

1) Generate an anchor cell line as outlined in Example 2.

2) Transfect bait probe pair into anchor cell line and select with 5 μg/ml blasticidin HCl+1 mg/ml zeocin until the cell line is stable.

3) Incubate cells with 100 nM of AP21967 for 2 hrs and stain for redistribution of bait probe to the membrane with myc antibody.

A. Robust membrane redistribution of bait probe: clone cells until cell line is homogenous. At this point, the cell line is ready for screening.

B. no membrane localization of bait probe: go to 4)

C. Membrane localization of bait probe in the absence of AP21967: repeat or try another anchor/bait system or orientation.

4) Stain for presence and localization of anchor with HA antibody.

A. Anchor is membrane localized: repeat all over or try another anchor/bait system or orientation.

B. Anchor is not membrane localized: repeat all over or try another anchor/bait system or orientation.

C. Anchor is not expressed: repeat all over.

The cell line generated at 3A above can be used for screening for novel partners of the bait protein (in this case GRB2) as detailed below:

1. Seed the screening cell line into 96 well plates at a density of 50000 cells/well. Incubate overnight under proper growth conditions as outlined in Example 2.

2. Transfect cDNA library from an ordered collection into the screening line. The library should contain a positive control (in this case Sos1). Incubate overnight to allow expression of cDNAs.

3. For each cDNA, detect the subcellular localization of the corresponding prey protein.

4. Induce dimerization by addition of 100 nM AP21967 and monitor the subcellular distribution of the prey proteins continuously until the positive control displays anchor type distribution.

5. Identify prey proteins that interact with the bait as those that change their subcellular distribution from a non-plasma membrane type distribution towards a plasma membrane type distribution.

6. Isolate the corresponding cDNAs by virtue of their position in the ordered collection of cDNAs.

### Example 6

Use of Protein Anchor Attached to the F-Actin Cytoskeleton and a FRB-FKBP Linker System to Screen for Interaction Inhibitors in the Extranuclear Cytoplasmic Compartment of Mammalian Cells.

The present example describes generic ways to produce cell lines suitable for screening compounds targeting a specific interaction between two partner components X and Y, where it is preferred that the interaction should be screened in the context of the extranuclear cytoplasmic compartment of the cell.

Two systems are described here, designed to be used together to discover compounds that specifically inhibit the interaction between the two partner components X and Y.

The first system consists of 2 parts, an anchor conjugated to FRB(T2098L) [plasmid construct ps1570] and a detectable conjugate comprising FKBP fused to EGFP [ps1208]—these conjugates are depicted in FIG. 4a. These two conjugates can be made to link together by the application of the dimerizer compound AP21967 (FIG. 4b). The 2-part system acts as a sorting assay for discarding compounds that may interfere with the linkage between FRB(T2098L) and FKBP that is formed by dimerizer compound AP21967, a rapamycin analog developed by ARIAD Pharmaceuticals. The 2-part assay also acts as counter screen for any compounds that may directly or indirectly affect the location of the anchor protein itself within the cell. FK506 is a suitable reference compound that competes against AP21967 for the binding site on FKBP, and can be used as a positive control to establish the maximum effect of interference compounds (Smax value) in this assay.

The second system is designed to run as the primary assay to find interaction inhibitors between any two partner proteins X and Y. As a generic description, this second system comprises 3 heterologous components, stably co-expressed within clonal CHO cells, these being an anchor conjugate, a mediator conjugate that could be conditionally dimerized to the anchor conjugate by AP21967, and a third detectable conjugate that contained EGFP. In this specific example, the 3 components of this second system were as follows:

1) The anchor conjugate was made by fusing the F-actin binding domain of α-actinin (amino acids 1-133 of full protein sequence) to FRB(T2098L) [ps1570] (also referred to as FRB*). The cellular localization of the anchor could be detected with an antibody directed against the HA-tag included in the anchor fusion protein

2) The mediator conjugate [ps1556] comprised wild-type FKBP protein fused to tandem repeats of FKBP(F36M), a mutant form of the protein that is known as F_{M} and also CAD, and the coding plasmid for which was obtained from ARIAD Pharmaceuticals

3) The detectable conjugate was made by fusing tandem repeats of CAD to EGFP [ps1547].

The three conjugates are depicted in diagrammatic form in FIG. 5a. CAD proteins spontaneously homodimerize, so mediator and detectable conjugates are normally linked together in the 3-part system (FIG. 5b). Therefore in this example, the protein interaction to be tested was the CAD:CAD link between mediator and detectable conjugates. Mediator and detectable conjugates can be made to link to the anchor conjugate through the application of dimerizer compound AP21967 (FIG. 5c). The link between CAD proteins can be broken by ARIAD compound AP21998 (FIG. 5d). AP21998 was therefore used as the reference compound to validate the system. CHO cells were transfected and cultured essentially as described in Example 2, except the complement of plasmids required for the 2 and 3-part systems were transfected simultaneously rather than sequentially (as described in Example 2).

For selection of co-expressing cells, 2-part system cells transfected with ps1570+ps1208 were cultured with 1 mg/ml zeocin+5 μg/ml blasticidin HCl. Cells of the 3-part system were cultured with 1 mg/ml zeocin+5 μg/ml blasticidin HCl+0.5 mg/ml G418 sulphate.

Once the cells were judged to be stably expressing their conjugates, generally after 10-14 days of culture under selection conditions post-transfection, the 2 and 3-part lines were checked for response to dimerizer compound by visual assessment of EGFP redistribution within the cell following treatment with AP21967. A positive redistribution response to AP21967 results in the appearance of bright aggregates of EGFP within the cytoplasm of both 2 and 3-part cell systems (FIGS. 6 and 7) within minutes of application of the compound to the cells. This redistribution response is robust and reversible, either by removal of AP21967 or by competition with FK506 (FIG. 22: EC₅₀ for FK506 versus 800 nM AP21967 is approximately 700 nM). In the 3-part system, the response can also be reversed by compound AP21998 (FIG. 7c).

Responding cells from both 2 and 3-part lines were selected and isolated from stable populations in the presence of dimerizer (typically using between 500 nM to 800 nM AP21967 in normal culture medium), and these cells grown up to form clonal colonies of cells. Clonal cultures were desirable to ensure a homogenous and uniform response to dimerizer and other treatments. Such properties yield the most useful response signals, with best signal to background and signal to noise characteristics. It was also possible to sort stable cell cultures using Fluorescence Activated Cell Sorting (FACS), using only the EGFP signal from cells as the sorting criterion. FACS'd cell cultures selected for highest EGFP expression also gave good responses to subsequent treatments with useful signal characteristics.

Without dimerizer present in the medium, cells from both 2- and 3-part ActinPaint systems display a more or less uniform distribution of GFP fluorescence, throughout cytoplasmic and nuclear compartments. In some cells of the 2-part system there are weakly distinguishable concentrations of fluorescence adjacent to the nucleus (FIG. 6a), and these may be due to entrapment or sequestering of the detectable conjugate in some enclosed compartment such as the Golgi body of the cell. Such structures did not label with probes specific for F-actin structures, such as rhodamine-labelled phalloidin (Molecular Probes Inc., Portland Oreg.), nor did they significantly degrade the final signal response of the system to AP21967 (FIG. 6b; 2-part ActinPaint cell line treated with 800 nM AP21967 for 60 minutes).

In the cells of the 3-part system a more noticeable structure or structures were visible in the cells prior to treatment with AP21967 (FIG. 7a). These were relatively faint compared to the intensely fluorescent aggregates that formed in the same locations after treatment with AP21967 (FIG. 7b). The aggregates stained with rhodamine-labelled phalloidin before (not shown) and after addition of dimerizer compound (FIG. 8a), indicating that they included F-actin. Since these faint spots are no longer evident when cells are treated with sufficient AP21998 compound to disperse the dimerizer-induced aggregates (FIG. 7c), it was concluded that they may represent a low level of spontaneous association between CAD domains and the anchored FRB* component. Again, these faint fluorescent aggregates in cells of the 3-part ActinPaint system did not significantly affect the dynamic range of the response to dimerizer.

As noted above, the aggregates of EGFP that formed in response to AP21967 in the ActinPaint lines were stained with rhodamine phalloidin to confirm the presence of F-actin in these structures. That the EGFP colocalised completely with F-Actin accumulations in these cells (FIGS. 8a and 8b) confirms that the ActinPaint-FRB* anchor system, in the presence of AP21967, is indeed able to sequester proteins fused to FKBP to the immobile F-actin cytoskeleton of the cell.

The number and brightness of EGFP aggregates in 2- and 3-part ActinPaint cell lines could be measured by any of the methods described in Example 3.

For compatibility with high throughput screening the cells were grown in microtitre plates for 16 hours from a seeding density of 1.0×10E5 cells per 400 μL, and an extraction procedure (described in Example 3) was used to remove mobile fluorescent components from cells, so that signal from immobile components could be measured. Plates were routinely stained with the nuclear dye Hoechst 22538 to enable correction of the immobile EGFP fluorescence signal from each well for cell density. A room temperature extraction buffer containing 0.1% to 0.4% formaldehyde in phosphate buffered saline (PBS, pH7.4)+0.1% Triton X-100 was found to give optimal signal to background in both the 2 and 3-part ActinPaint lines when applied for 10 minutes, prior to full fixation and nuclear staining with 4% formaldehyde+10 μM Hoechst 22538 for a further 10 minutes, followed by 3 wash steps using PBS. In cells treated with AP21967, the extraction procedure left immobile EGFP-labelled aggregates anchored to F-actin within the remains of the extracted cells (FIG. 9).

Using the extraction procedure on cells in microtitre plates a dose response of the ActinPaint systems to AP21967 was generated (FIG. 10 shows the response of the 3-part ActinPaint system). Cells were treated in HAM F12 growth medium+10% FCS to various concentrations of AP21967 for 2 hours, then mobile EGFP-labelled components extracted as described. Signals from immobile EGFP-labelled components and from the nuclear stain were read on an fluorescence plate reader (Fluoroskan Ascent CF, Labsystems, Finland) equipped with appropriate filter sets (EGFP: excitation 485 nm, emission 527 nm; Hoechst 22538: excitation 355 nM, emission 460 nm). The response of the 3-part line to AP21967 did not reach a maximum over the range of AP21967 concentrations used in FIG. 10, but for both 2 and 3-part systems using the extraction procedure described, a concentration of 1000 nM AP21967 increased the signal from immobile EGFP-labelled components remaining in the cells by approximately 3-fold relative to untreated cells. For further experiments a dimerizer concentration of 800 nM for 2 hours was selected as giving adequate signal to background for extracted cells to be read on the Ascent plate reader.

Although 2 hours incubation with AP21967 was used as standard for these tests, the response to dimerizer of both 2- and 3-part systems was actually very much faster. At 37° C., formation of EGFP-bright aggregates in the cells of the 2-part system was apparent after only 2 minutes exposure to 800 nM AP21967, and reached a maximum after approximately 15 minutes (FIG. 11). The 3-part system was a little slower in its response to AP21967, but EGFP labelling of aggregates was still clearly visible after only 15 minutes exposure to 800 nM dimerizer compound, and reached a maximum after approximately 40 minutes (FIG. 12).

To demonstrate that the ActinPaint system can be used to find interaction inhibitor compounds, AP21998 was used to break the links between CAD domains that connect the detectable conjugates to the mediator conjugates as they join the F-actin anchored aggregates. Dose-response of this CAD-interaction inhibitor in the 3-part ActinPaint system could be compared with the compound's efficacy and potency in other systems, to determine if similar sensitivity to the inhibitor (and other inhibitors) could be expected from the

ActinPaint system. Cells of the 3-part system were grown as described in microtitre plates and treated for 2 hours with a mixture of 800 nM AP21967 plus various concentrations of AP21998 then incubated at 37° C. for 2 hours. Plates were processed for extraction and nuclear staining then read in an Ascent plate reader. Results are shown in FIG. 13, corrected for background and cell number. The EC₅₀ for AP21998 was approximately 1.1 μM for the removal of EGFP-labelled components from the F-actin aggregates. After treatment with concentrations of AP21998 greater than 5 μM, no EGFP-label remained on the aggregates. However, the F-actin aggregates themselves could still be detected by rhodamine-labelled phalloidin, and both the ActinPaint anchor conjugate and mediator conjugate were still attached to the F-actin aggregates, as could be demonstrated by antibody detection of the HA and V5 epitopes respectively present in these constructs. The AP21998 compound stripped away only the CAD.CAD-EGFP (detectable) conjugate from the F-actin aggregates. Data supplied by ARIAD Pharmaceuticals (RPD™ Regulated Secretion/Aggregation Kit fact sheet, Version 2.0, published at www.ariad.com) indicate that the AP21998 compound has an EC₅₀ activity in a transcription factor-based detection system of approximately 0.2 μM. It is therefore concluded that the use of the ActinPaint anchor does not significantly affect the ability of protein interaction inhibitors to break interactions tethered to that anchor by means of an intermediate FRB*-FKBP linkage.

A useful indicator of the suitability of an assay for HTS is the so called Z-factor (Zhang J H, Chung T D, Oldenburg K R. (1999) A Simple Statistical Parameter for Use in Evaluation and Validation of High Throughput Screening Assays. J Biomol Screen.;4(2):67-73). Mean and standard deviation (sd) are calculated from raw data for positive and negative control wells to which no dimerizer has been added (So, 8 wells) and wells to which only 800 nM AP21967 has been added Smax (8 wells) and the Z-factor calculated as follows:

Z=1−(3*sd[So]+3*sd[Smax])/(Mean[Smax]−Mean[So])

The Z-factor is a simple statistical parameter of assay validity and can be used to assess the reliability of each datapoint (Sn) produced by the assay versus the percent activity it registers, where percent activity is 100*(Sn−So)/(Smax−So).

The data used to produce FIG. 13 yield a Z-factor of 0.57. Typical HTS assays require a Z-factor of greater than 0.3. The ActinPaint iGRIP assay run with extraction procedure and read on the Ascent plate reader therefore qualifies as being compatible with HTS methods as regards Z-factor.

The 3-part ActinPaint system is a useful generic method by which to screen for compounds that inhibit protein interactions, and is of especial use when it is desirable or preferred to screen for such compounds in the extranuclear cytoplasmic compartment of mammalian cells.

### Example 7

Use of a DNA Associated Protein Anchor and a FRB-FKBP Linker System to Screen for Interaction Inhibitors in the Nuclear Compartment of Mammalian Cells.

The present example describes generic ways to produce cell lines suitable for screening compounds targeting a specific interaction between two partner components X and Y, where it is preferred that the interaction should be screened in the context of the nuclear compartment of the cell.

Two systems are described here, designed to be used together to discover compounds that specifically inhibit the interaction between the two partner components X and Y.

The first system consists of 2 parts, an anchor conjugated to FRB(T2098L) [plasmid construct ps1569] and a detectable conjugate comprising FKBP fused to EGFP [ps1208]. The 2-part system acts as a sorting assay for discarding compounds that may interfere with the linkage between FRB(T2098L) and FKBP that is formed by dimerizer compound AP21967, a rapamycin analog developed by ARIAD Pharmaceuticals. The 2-part assay also acts as counter screen for any compounds that may directly or indirectly affect the location of the anchor protein itself within the cell.

The second system is designed to run as the primary assay to find interaction inhibitors between any two partner proteins X and Y. As a generic description, this second system comprises 3 heterologous components, stably co-expressed within clonal CHO cells, these being an anchor conjugate, a mediator conjugate that could be conditionally dimerized to the anchor conjugate by AP21967, and a third detectable conjugate that contained EGFP. In this specific example, the 3 components of this second system were as follows:

1) The anchor conjugate was made by fusing histone H2B to FRB(T2098L) [ps1569] (also referred to as FRB*). The cellular localization of the anchor could be detected with an antibody directed against the HA-tag included in the anchor fusion protein

2) The mediator conjugate [ps1556] comprised wild-type FKBP protein fused to tandem repeats of FKBP(F36M), a mutant form of the protein that is known as F_{M} and also CAD, and the coding plasmid for which was obtained from ARIAD Pharmaceuticals

3) The detectable conjugate was made by fusing tandem repeats of CAD to EGFP [ps1547].

The three conjugates are depicted in diagrammatic form in FIG. 5a. CAD proteins spontaneously homodimerize, so mediator and detectable conjugates are normally linked together in the 3-part system (FIG. 5b). Therefore in this example, the protein interaction to be tested was the CAD:CAD link between mediator and detectable conjugates. Mediator and detectable conjugates can be made to link to the anchor conjugate through the application of dimerizer compound AP21967 (FIG. 5c). The link between CAD proteins can be broken by ARIAD compound AP21998 (FIG. 5d). AP21998 was therefore used as the reference compound to validate the system.

CHO cells were transfected and cultured essentially as described in Example 2, except the complement of plasmids required for the 2 and 3-part systems were transfected simultaneously rather than sequentially (as described in Example 2).

For selection of co-expressing cells, 2-part system cells transfected with ps1569+ps1208 were cultured with 1 mg/ml zeocin+5 μg/ml blasticidin HCl. Cells of the 3-part system were cultured with 1 mg/ml zeocin+5 μg/ml blasticidin HCl+0.5 mg/ml G418 sulphate.

Once the cells were judged to be stably expressing their conjugates, generally after 10-14 days of culture under selection conditions post-transfection, the 2 and 3-part lines were checked for response to dimerizer compound by visual assessment of EGFP redistribution within the cell following treatment with AP21967. A positive redistribution response to AP21967 results in the increase of EGFP fluorescence in the nuclei of both 2 and 3-part cell systems (FIGS. 14 and 15) within minutes of application of the compound to the cells. This redistribution response to dimerizer is robust and can be competed by FK506. In the 3-part system, the response can also be competed by compound AP21998 (FIG. 15c).

Responding cells from both 2 and 3-part lines were selected and isolated from stable populations in the presence of dimerizer (typically using between 500 nM to 800 nM AP21967 in normal culture medium), and these cells grown up to form clonal colonies of cells. Clonal cultures were desirable to ensure a homogenous and uniform response to dimerizer and other treatments. Such properties yield the most useful response signals, with best signal to background and signal to noise characteristics. It was also possible to sort stable cell cultures using Fluorescence Activated Cell Sorting (FACS), using only the EGFP signal from cells as the sorting criterion. FACS'd cell cultures selected for highest EGFP expression also gave good responses to subsequent treatments with useful signal characteristics.

Without dimerizer present in the medium, cells from both 2- and 3-part Histone H2B systems display a more or less uniform distribution of GFP fluorescence, throughout cytoplasmic and nuclear compartments (FIGS. 14a and 15a). Dimerizer addition results in recruitment of the CAD-linked mediator+detectable complexes to the nuclear compartment (FIGS. 14b and 15b). The overall effect of this recruitment process is to deplete the cytoplasmic compartment of EGFP-labelled components, simultaneously increasing the concentration of EGFP-labelled components in the nucleus of each cell.

Cells of the 2 and 3-part Histone H2B lines treated with AP21967 show a clear colocalisation of the EGFP fluorescence with the Histone H2B-FRB* anchor component. The exclusively nuclear location of the Histone H2B-FRB* anchor is shown in FIG. 16a, here labelled with a primary anti-HA antibody (HA.11 mouse monoclonal antibody, Covance Inc, New Jersey, USA) and detected with a fluorescently labelled anti-mouse secondary antibody (AlexaFluor 546 goat anti-mouse, Molecular Probes Inc., Portland, Oreg., USA). In the same (responding) cells, the EGFP fluorescence colocalises with the HA tag (FIG. 16b).

Recruitment of the EGFP-labelled components from cytoplasm to nucleus in 2- and 3-part Histone H2B cell lines could be measured by any of the methods described in Example 3.

For compatibility with high throughput screening the cells were grown in microtitre plates for 16 hours from a seeding density of 1.0×10E5 cells per 400 μL, and an extraction procedure (described in Example 3) was used to remove mobile fluorescent components from cells, so that signal from immobile components locked in the nucleus could be measured. Plates were routinely stained with the nuclear dye Hoechst 22538 to enable correction of the immobile EGFP fluorescence signal from each well for cell density. A room temperature extraction buffer containing 0.1% formaldehyde in phosphate buffered saline (PBS, pH7.4)+0.1% Triton X-100 was found to give optimal signal to background in both the 2 and 3-part Histone H2B lines when applied for 10 minutes, prior to full fixation and nuclear staining with 4% formaldehyde+10 μM Hoechst 22538 for a further 10 minutes, followed by 3 wash steps using PBS. In cells treated with AP21967, the extraction procedure left immobile EGFP-labelled components anchored in nuclei within the remains of the extracted cells (FIG. 17).

Using the extraction procedure on cells in microtitre plates a dose response of the Histone H2B systems to AP21967 was generated (FIG. 18 shows the response of the 3-part Histone H2B system). Cells were treated in HAM F12 growth medium+10% FCS to various concentrations of AP21967 for 2 hours, then mobile EGFP-labelled components extracted as described. Signals from immobile EGFP-labelled components and from the nuclear stain were read on an fluorescence plate reader (Fluoroskan Ascent CF, Labsystems, Finland) equipped with appropriate filter sets (EGFP: excitation 485 nm, emission 527 nm; Hoechst 22538: excitation 355 nM, emission 460 nm). The response of the 3-part line to AP21967 did not reach a maximum over the range of AP21967 concentrations used in FIG. 18, but for both 2 and 3-part systems using the extraction procedure described, a concentration of 1000 nM AP21967 increased the signal from immobile EGFP-labelled components remaining in the cells by approximately 2-fold relative to untreated cells. For further experiments a dimerizer concentration of 800 nM for 2 hours was selected as giving adequate signal to background for extracted cells to be read on the Ascent plate reader.

Although 2 hours incubation with AP21967 was used as standard for these tests, the response to dimerizer of both 2- and 3-part systems was actually very much faster. At 37° C., recruitment of EGFP-labelled components in the nuclei of the 2-part system was apparent after only 2 minutes exposure to 800 nM AP21967, and reached a maximum after approximately 10 minutes (FIG. 19). The 3-part system was a little slower in its response to AP21967, but EGFP labelling of aggregates was still clearly visible after only 15 minutes exposure to 800 nM dimerizer compound, and reached a maximum after approximately 40 minutes (FIG. 20).

To demonstrate that the Histone H2B system can be used to find interaction inhibitor compounds, AP21998 was used to break the links between CAD domains that connect the detectable conjugates to the mediator conjugates as they anchor in the nuclei. Dose-response of this CAD-interaction inhibitor in the 3-part Histone H2B system could be compared with the compound's efficacy and potency in other systems, to determine if similar sensitivity to the inhibitor (and other inhibitors) could be expected from the Histone H2B system. Cells of the 3-part system were grown as described in microtitre plates and treated for 2 hours with a mixture of 800 nM AP21967 plus various concentrations of AP21998 then incubated at 37° C. for 2 hours. Plates were processed for extraction and nuclear staining then read in an Ascent plate reader. Results are shown in FIG. 21, corrected for background and cell number. The EC₅₀ for AP21998 was approximately 1.8 μM for the removal of EGFP-labelled components from the cell nuclei. After treatment with concentrations of AP21998 greater than 5 μM, no EGFP-label remained in the nuclei. However, the both the Histone H2B anchor conjugate and mediator conjugate were still located to the nuclei, as could be demonstrated by antibody detection of the HA and V5 epitopes respectively present in these constructs. The AP21998 compound stripped away only the CAD.CAD-EGFP (detectable) conjugate from the nuclei. Data supplied by ARIAD Pharmaceuticals (RPD™ Regulated Secretion/Aggregation Kit fact sheet, Version 2.0, published at www.ariad.com) indicate that the AP21998 compound has an EC₅₀ activity in a transcription factor-based detection system of approximately 0.2 μM. It is therefore concluded that the use of the Histone H2B anchor does not significantly affect the ability of protein interaction inhibitors to break interactions tethered to that anchor by means of an intermediate FRB*-FKBP linkage.

A useful indicator of the suitability of an assay for HTS is the so called Z-factor (Zhang J H, Chung T D, Oldenburg K R. (1999) A Simple Statistical Parameter for Use in Evaluation and Validation of High Throughput Screening Assays. J Biomol Screen.; 4(2):67-73). Mean and standard deviation (sd) are calculated from raw data for positive and negative control wells to which no dimerizer has been added (So, 8 wells) and wells to which only 800 nM AP21967 has been added Smax (8 wells) and the Z-factor calculated as follows:

Z=1−(3*sd[So]+3*sd[Smax])/(Mean[Smax]−Mean[So])

The Z-factor is a simple statistical parameter of assay validity and can be used to assess the reliability of each datapoint (Sn) produced by the assay versus the percent activity it registers, where percent activity is 100*(Sn−So)/(Smax−So).

The data used to produce FIG. 21 yield a Z-factor of 0.52. Typical HTS assays require a Z-factor of greater than 0.3. The Histone H2B iGRIP assay run with extraction procedure and read on the Ascent plate reader therefore qualifies as being compatible with HTS methods as regards Z-factor.

The 3-part Histone H2B system is a useful generic method by which to screen for compounds that inhibit protein interactions, and is of especial use when it is desirable or preferred to screen for such compounds in the nuclear compartment of mammalian cells.

### Example 8

Use of a Plasma Membrane Associated Protein Anchor and a FRB*-FKBP Linker System to Screen for Interaction Inhibitors at the Plasma Membrane of Mammalian Cells.

The present example describes generic ways to produce cell lines suitable for screening compounds targeting a specific interaction between two partner components X and Y, where it is preferred that the interaction should be screened in the context of a plasma membrane location in the cell.

Two systems are described here, designed to be used together to discover compounds that specifically inhibit the interaction between the two partner components X and Y.

The first system consists of 2 parts, an anchor conjugated to FRB(T2098L) [plasmid construct ps1549] and a detectable conjugate comprising FKBP fused to EGFP [ps1208]. The 2-part system acts as a sorting assay for discarding compounds that may interfere with the linkage between FRB(T2098L) and FKBP that is formed by dimerizer compound AP21967, a rapamycin analog developed by ARIAD Pharmaceuticals. The 2-part assay also acts as counter screen for any compounds that may directly or indirectly affect the location of the anchor protein itself within the cell.

The second system is designed to run as the primary assay to find interaction inhibitors between any two partner proteins X and Y. As a generic description, this second system comprises 3 heterologous components, stably co-expressed within clonal CHO cells, these being an anchor conjugate, a mediator conjugate that could be conditionally dimerized to the anchor conjugate by AP21967, and a third detectable conjugate that contained EGFP. In this specific example, the 3 components of this second system were as follows:

1) The anchor conjugate was made by fusing c-Src(1-14) to FRB(T2098L) [ps1549]. The cellular localization of the anchor could be detected with an antibody directed against the HA-tag included in the anchor fusion protein

2) The mediator conjugate [ps1556] comprised wild-type FKBP protein fused to tandem repeats of FKBP(F36M), a mutant form of the protein that is known as F_{M} and also CAD, and the coding plasmid for which was obtained from ARIAD Pharmaceuticals

3) The detectable conjugate was made by fusing tandem repeats of CAD to EGFP [ps1547].

The three conjugates are depicted in diagrammatic form in FIG. 5a. CAD proteins spontaneously homodimerize, so mediator and detectable conjugates are normally linked together in the 3-part system (FIG. 5b). Therefore in this example, the protein interaction to be tested was the CAD:CAD link between mediator and detectable conjugates. Mediator and detectable conjugates can be made to link to the anchor conjugate through the application of dimerizer compound AP21967 (FIG. 5c). The link between CAD proteins can be broken by ARIAD compound AP21998 (FIG. 5d). AP21998 was therefore used as the reference compound to validate the system.

CHO cells were transfected and cultured essentially as described in Example 2, except the complement of plasmids required for the 2 and 3-part systems were transfected simultaneously rather than sequentially (as described in Example 2).

For selection of co-expressing cells, 2-part system cells transfected with ps1549+ps1208 were cultured with 1 mg/ml zeocin+5 μg/ml blasticidin HCl. Cells of the 3-part system were cultured with 1 mg/ml zeocin+5 μg/ml blasticidin HCl+0.5 mg/ml G418 sulphate.

Once the cells were judged to be stably expressing their conjugates, generally after 10-14 days of culture under selection conditions post-transfection, the 2 and 3-part lines were checked for response to dimerizer compound by visual assessment of EGFP redistribution within the cell following treatment with AP21967. A positive redistribution response to AP21967 results in the increase of EGFP fluorescence at the plasma membrane of both 2 and 3-part cell systems (Figures ** and **) within minutes of application of the compound to the cells. This redistribution response to dimerizer is robust and can be competed by FK506. In the 3-part system, the response can also be competed by compound AP21998.

Responding cells from both 2 and 3-part lines were selected and isolated from stable populations in the presence of dimerizer (typically using between 500 nM to 800 nM AP21967 in normal culture medium), and these cells grown up to form clonal colonies of cells. Clonal cultures were desirable to ensure a homogenous and uniform response to dimerizer and other treatments. Such properties yield the most useful response signals, with best signal to background and signal to noise characteristics. It was also possible to sort stable cell cultures using Fluorescence Activated Cell Sorting (FACS), using only the EGFP signal from cells as the sorting criterion. FACS'd cell cultures selected for highest EGFP expression also gave good responses to subsequent treatments with useful signal characteristics.

Without dimerizer present in the medium, cells from both 2- and 3-part Src(1-14) systems display a more or less uniform distribution of GFP fluorescence, throughout cytoplasmic and nuclear compartments (FIGS. 23a and 24a). Dimerizer addition results in recruitment of the CAD-linked mediator+detectable complexes to the plasma membrane (FIGS. 23b and 24b). The overall effect of this recruitment process is to deplete the cytoplasmic compartment of EGFP-labelled components, simultaneously increasing the concentration of EGFP-labelled components at the plasma membrane of each cell.

Recruitment of the EGFP-labelled components from cytoplasm to nucleus in 2- and 3-part Src(1-14) cell lines could be measured by any of the methods described in Example 3. Measurement on the FLIPR plate reader with addition of a fluorescence quenching agent such as trypan blue is the preferred method of signal extraction.

Although 2 hours incubation with AP21967 was used as standard for these tests, the response to dimerizer of both 2- and 3-part systems was actually very much faster. The 3-part Histone Src(1-14) system is a useful generic method by which to screen for compounds that inhibit protein interactions, and is of especial use when it is desirable or preferred to screen for such compounds at the plasma membrane of mammalian cells.

1. A method for detecting if a compound disrupts the interaction between two intracellular proteins comprising the steps of:
(a) providing a cell that contains three heterologous conjugates,
a first heterologous conjugate comprising an anchor protein that specifically binds to an internal structure within the cell conjugated to an interactor protein of type A
a second heterologous conjugate comprising an interactor protein of type B conjugated to the first protein of interest
a third heterologous conjugate comprising a second protein of interest conjugated to a detectable group,
(b) inducing interaction of protein of type A with protein of type B through application of a dimerizer molecule;
(c) detecting the intracellular distribution of the detectable group
an intracellular distribution of said detectable group mimicking the intracellular distribution of the anchor-protein being indicative of binding between the two proteins of interest;
(d) repeating step (c) with and without the compound;
a change in intracellular distribution of the detectable group with and without the compound being indicative that the compound disrupts the interaction between the first and the second protein of interest.

2. A method for detecting if a compound induces interaction between two intracellular proteins comprising the steps of:
(a) providing a cell that contains three heterologous conjugates,
a first heterologous conjugate comprising an anchor protein that specifically binds to an internal structure within the cell conjugated to an interactor protein of type A
a second heterologous conjugate comprising an interactor protein of type B conjugated to the first protein of interest
a third heterologous conjugate comprising a second protein of interest conjugated to a detectable group,
(b) inducing interaction of protein of type A with protein of type B through application of a dimerizer molecule;
(c) detecting the intracellular distribution of the detectable group
(d) repeating step (c) with and without the compound;
an intracellular distribution of said detectable group mimicking the intracellular distribution of the anchor-protein;
a change in intracellular distribution of the detectable group with the compound to being indicative that the compound induces interaction between the first and the second protein of interest.

3. A method for screening for compounds modulating the interaction between two intracellular proteins comprising the steps of:
(a) providing a cell that contains three heterologous conjugates,
a first heterologous conjugate comprising an anchor protein that specifically binds to an internal structure within the cell conjugated to an interactor protein of type A
a second heterologous conjugate comprising an interactor protein of type B conjugated to the first protein of interest
a third heterologous conjugate comprising a second protein of interest conjugated to a detectable group,
(b) detecting the non-dimerized intracellular distribution of the detectable group;
(c) inducing interaction of protein of type A with protein of type B through application of a dimerizer molecule;
(d) detecting the dimerized intracellular distribution of the detectable group;
(e) applying the test compound
(f) detecting the intracellular distribution of the detectable group in the test-stage;
the non-dimerized distribution forming the first, and the dimerized distribution forming the second end of the detection scale on which the intracellular distribution in the test-stage is measured such that if the intracellular distribution in the test-stage mimics the dimerized distribution, the compound has no effect on the interaction between the two proteins of interest, and if the intracellular distribution in the test-stage mimics the non-dimerized distribution the compound disrupts the interaction between the two proteins of interest.

4. A method for screening for compounds modulating the interaction between two intracellular proteins comprising the steps of:
(a) providing at least three homogenous groups of homogenous cells that contains three heterologous conjugates,
a first heterologous conjugate comprising an anchor protein that specifically binds to an internal structure within the cell conjugated to an interactor protein of type A
a second heterologous conjugate comprising an interactor protein of type B conjugated to the first protein of interest
a third heterologous conjugate comprising a second protein of interest conjugated to a detectable group;
(b) applying the test compound to the first group of cells;
(c) inducing interaction of protein of type A with protein of type B through application of a dimerizer molecule to all three groups of cells;
(d) applying a competitive reference compound to the second group of cells;
(e) detecting the intracellular distribution of the detectable group in the test-stage in the first one;
(f) detecting the non-dimerized intracellular distribution of the detectable group in the second group;
(g) detecting the dimerized intracellular distribution of the detectable group in the third group;
the non-dimerized distribution forming the first, and the dimerized distribution forming the second end of the detection scale on which the intracellular distribution in the test-stage is measured such that if the intracellular distribution in the test-stage mimics the dimerized distribution, the compound has no effect on the interaction between the two proteins of interest, and if the intracellular distribution in the test-stage mimics the non-dimerized distribution the compound disrupts the interaction between the two proteins of interest.

5. A method for identifying novel interaction partners for a bait protein comprising the steps of:
(a) providing a cell line where each cell contains two heterologous conjugates,
the first heterologous conjugate comprising an anchor protein that can specifically bind to an internal structure within the cell conjugated to an interactor protein of class A
the second heterologous conjugate comprising an interactor protein of class B conjugated to the bait protein
(b) introducing into said cell line a cDNA library coding for prey proteins conjugated to a detectable group
(c) detecting the intracellular distribution of the detectable group
(d) inducing interaction of protein of type A with protein of type B through application of a dimerizer molecule
(e) detecting the intracellular distribution of the detectable group
the intracellular distribution of said detectable group mimicking the intracellular distribution of the anchor-protein only in the presence of said dimerizer molecule being indicative of binding between the bait and prey proteins
(f) isolating prey conjugates that show indication of binding to the bait component.

6. A method according to claim 1, wherein the anchor protein is a protein containing the transmembrane domain of the epidermal growth factor receptor (EGFR), or containing the transmembrane domain of one of the integrin protein family, or containing the myristoylation sequence from c-Src (residues 1-14).

7. A method according to claim 1, wherein the anchor protein is a histone protein or a protein normally restricted to nucleoli, for example the p120 nucleolar protein.

8. A method according to claim 1, wherein the anchor protein is a protein normally confined to mitochondrial outer or inner membranes for example VDAC, F₀ subunit of ATP-ase, or NADH dehydrogenase.

9. A method according to claim 1, wherein the anchor protein is a protein normally confined to the various different regions of Golgi bodies for example TGN38 or ADAM12-L.

10. A method according to claim 1, wherein the anchor protein is a protein normally confined to focal adhesion complexes for example P125, FAK, integerin alpha or beta, or paxillin.

11. A method according to claim 1, wherein the anchor protein is a protein normally associated with cytoskeletal structures such as F-actin strands or micro tubular bundles for example MAP4, actin binding domain of alpha-actinin, kinesins, myosins or dyniens.

12. A method according to claim 1, wherein the anchor protein is a protein normally associated with nuclear material or nuclear components, such as histone proteins, including histones H1, H2A, H2B, H3, H4, and variants thereof.

13. A method according to claim 1, wherein the anchor protein is a protein normally associated with nuclear membrane such as A and B type lamins, or associated with nuclear bodies such as splicing bodies, Cajal bodies, PML nuclear bodies (PML oncogenic domains, PODS), or transcription engines such as RNA polymerase POL-II.

14. A method according to claim 1, wherein linker protein A is FKBP12, linker protein B is FRAP (or vise versa) and the dimerizer is Rapamycin.

15. A method according to claim 1, wherein linker protein A is FKBP12, linker protein B is FRB (T2098L) (or vise versa) and the dimerizer is Rapamycin.

16. A method according to claim 1, wherein linker protein A is FKBP12, linker protein B is FRB (T2098L) (or vise versa) and the dimerizer is AP21967.

17. A method according to claim 1, wherein linker protein A is FKBP12, linker protein B is FKBP12 (or vise versa) and the dimerizer is AP21967.

18. A method according to claim 1, wherein linker protein A is FKBP12, linker protein B is FKBP12 (or vise versa) and the dimerizer is AP21967.

19. A method according to claim 1, wherein linker protein A is FKBP12, linker protein B is Calcineurin (or vise versa) and the dimerizer is FK506.

20. A method according to claim 1, wherein the interactor A and B are steroid hormone receptors and the dimerizing agent is the cognate hormone ligand.

21. A method according to claim 20, wherein the interactor protein A and B are estrogen receptors and the dimerizer agent is estrogen.

22. A method according to claim 1, wherein interactor protein A is the full-length or the ligand-binding domain of a steroid hormone receptor and interactor protein B is chosen among the family of steroid hormone receptor co-activators including, but not limited to, SRC-1, GRIP-1, ACTR, AIB-1 and the dimerizer molecule is the cognate hormone.

23. A method according to claim 1, wherein the interactor protein A and B are F36V mutated FKBP12 and FK506and/or Rapamycin are used as inhibitors of dimerizing.

24. A method according to claim 1, wherein one of interactor A or B protein is FKBP12 and the other of interactor A or B protein is type I TGF-beta receptor and FK506 and/or Rapamycin are used as inhibitors of dimerizing.

25. A method according to claim 1, wherein a full-length or ligand-binding domain only nuclear hormone receptor including, but not limited to, the thyroid hormone receptor or the retinoid acid receptor is chosen as one of interactor A or B and a full-length (or fragment thereof) nuclear hormone co-repressor such as, but not limited to, N-CoR or SMRT as the other of interactor A or B, and in each case cognate hormone is used as dimerization inhibitor.

26. A method according to claim 1, wherein the detectable group is a Green Fluorescent Protein (GFP).

27. A method according to claim 1, wherein the GFP is a GFP wherein the amino acid in position 1 upstream from the chromophore has been mutated to provide an increase of fluorescence intensity when the fluorescent protein of the invention is expressed in cells.

28. A method according to claim 1, wherein the GFP has an F64L mutation.

29. A method according to claim 1, wherein the GFP is a GFP variant selected from the group consisting of F64L-GFP, F64L-Y66H-GFP, F64L-S65T-GFP, F64L-E222G-GFP and EGFP.

30. A method according to claim 4, wherein the cDNA library is produced as an ordered collection and introduced into the bait cell line by High Throughput transfection

31. A method according to claim 4, wherein the cDNA library is introduced into the bait cell line by transfection followed by selection, such as by fluorescence associated cell sorting or FACS.

32. A cell comprising three heterologous conjugates
a first heterologous conjugate comprising an anchor protein that specifically binds to an internal structure within the cell conjugated to an interactor protein of type A
a second heterologous conjugate comprising an interactor protein of type B conjugated to the first protein of interest
a third heterologous conjugate comprising a second protein of interest conjugated to a detectable group.

33. A cell according to claim 32, wherein the anchor protein is a histone protein or a protein normally restricted to nucleoli, for example the p120 nucleolar protein.

34. A cell according to claim 32, wherein the anchor protein is a protein normally confined to mitochondrial outer or inner membranes for example VDAC, F₀ subunit of ATP-ase, or NADH dehydrogenase.

35. A cell according to claim 32, wherein the anchor protein is a protein normally confined to the various different regions of Golgi bodies for example TGN38 or ADAM12-L.

36. A cell according to claim 32, wherein the anchor protein is a protein normally confined to focal adhesion complexes for example P125, FAK, integerin alpha or beta, or paxillin.

37. A cell according to claim 32, wherein the anchor protein is a protein normally associated with cytoskeletal structures such as F-actin strands or micro tubular bundles for example MAP4, actin binding domain of alpha-actinin, kinesins, myosins or dyniens.

38. A cell according to claim 32, wherein the anchor protein is a protein normally associated with nuclear material or nuclear components, such as histone proteins, including histones H1, H2A, H2B, H3, H4, and variants thereof.

39. A cell according to claim 32, wherein the anchor protein is a protein normally associated with nuclear membrane such as A and B type lamins, or associated with nuclear bodies such as splicing bodies, Cajal bodies, PML nuclear bodies (PML oncogenic domains, PODS), or transcription engines such as RNA polymerase POL-II.

40. A cell according to claim 32, wherein linker protein A is FKBP12, linker protein B is FRAP (or vise versa) and the dimerizer is Rapamycin.

41. A cell according to claim 32, wherein linker protein A is FKBP12, linker protein B is FRB (T2098L) (or vise versa) and the dimerizer is Rapamycin.

42. A cell according to claim 32, wherein linker protein A is FKBP12, linker protein B is FRB (T2098L) (or vise versa) and the dimerizer is AP21967.

43. A cell according to claim 32, wherein linker protein A is FKBP12, linker protein B is FKBP12 (or vise versa) and the dimerizer is AP21967.

44. A cell according to claim 32, wherein linker protein A is FKBP12, linker protein B is FKBP12 (or vise versa) and the dimerizer is AP21967.

45. A cell according to claim 32, wherein linker protein A is FKBP12, linker protein B is Calcineurin (or vise versa) and the dimerizer is FK506.

46. A cell according to claim 32, wherein the interactor A and B are steroid hormone receptors and the dimerizing agent is the cognate hormone ligand.

47. A cell according to claim 32, wherein the interactor protein A and B are estrogen receptors and the dimerizer agent is estrogen.

48. A cell according to claim 32, wherein interactor protein A is the full-length or the ligand-binding domain of a steroid hormone receptor and interactor protein B is chosen among the family of steroid hormone receptor co-activators including, but not limited to, SRC-1, GRIP-1, ACTR, AIB-1 and the dimerizer molecule is the cognate hormone.

49. A cell according to claim 32, wherein the interactor protein A and B are F36V mutated FKBP12 and FK506 and/or Rapamycin are used as inhibitors of dimerizing.

50. A cell according to claim 32, wherein one of interactor A or B protein is FKBP12 and the other of interactor A or B protein is type I TGF-beta receptor and FK506 and/or Rapamycin are used as inhibitors of dimerizing.

51. A cell according to claim 32, wherein a full-length or ligand-binding domain only nuclear hormone receptor including, but not limited to, the thyroid hormone receptor or the retinoid acid receptor is chosen as one of interactor A or B and a full-length (or fragment thereof) nuclear hormone co-repressor such as, but not limited to, N-CoR or SMRT as the other of interactor A or B, and in each case cognate hormone is used as dimerization inhibitor.

52. A cell according to claim 32, wherein the detectable group is a Green Fluorescent Protein (GFP).

53. A cell according to claim 32, wherein the GFP is a GFP wherein the amino acid in position 1 upstream from the chromophore has been mutated to provide an increase of fluorescence intensity when the fluorescent protein of the invention is expressed in cells.

54. A cell according to claim 32, wherein the GFP has an F64L mutation.

55. A cell according to claim 32, wherein the GFP is a GFP variant selected from the group consisting of F64L-GFP, F64L-Y66H-GFP, F64L-S65T-GFP, F64L-E222G-GFP and EGFP.

56. A cell comprising the first heterologous conjugate according to claim 1.

57. A cell comprising the first and the second heterologous conjugates according to claim 1.

58. A method according to claim 1 further comprising a counter screen comprising the steps of:
(v) providing a cell that contain two heterologous conjugates,
a first heterologous conjugate comprising an anchor protein that specifically binds to an internal structure within the cell conjugated to an interactor protein of type A
a second heterologous conjugate comprising an interactor protein of type B conjugated to a detectable group
(vi) inducing interaction of protein of type A with protein of type B through application of a dimerizer molecule;
(vii) detecting the intracellular distribution of the detectable group
an intracellular distribution of said detectable group mimicking the intracellular distribution of the anchor-protein being indicative of binding between the protein of type A and the protein of type B
(viii) repeating step (iii) with and without the compound found to disrupt the binding between the two proteins of interest;
a change in intracellular distribution of the detectable group with and without said compound found to disrupt the binding between the two proteins of interest being indicative that the compound is a false positive capable of disrupting the binding between protein of type A and protein of type B.

59. A method according to claim 1 configured for High Throughput Screening further comprising the steps of:
(i) adding extraction buffer to the cells, the extraction buffer comprising a cellular fixation agent and a cellular permeabilization agent; and
(ii) measuring the light emitted from cells of step (i).

60. A nucleic acid encoding the first heterologous conjugate according to claim 1.

61. A nucleic acid encoding the second heterologous conjugate according to claim 1.

62. A nucleic acid encoding the third heterologous conjugate according to claim 1.

63. A kit comprising a cell comprising the first heterologous conjugate, a nucleic acid encoding the second heterologous conjugate and a nucleic acid encoding the third heterologous conjugate.

64. A kit comprising three pieces of nucleic acid encoding each of the heterologous conjugates according to claim 1.


SEQUENCE LISTING (12 sequences)

SEQ ID NO: 1 - DNA, 1452 bp, Aequoria Victoria and Human
  CDS (1)..(1452)
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagaggagtgcaggtggaaaccatctccccgggagacgggcgcaccttccccaagcgcggccagacctgcgtggtgcactacaccgggatgcttgaagatggaaagaaaatggattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggagtgcaggtggaaaccatctccccgggagacgggcgcaccttccccaagcgcggccagacctgcgtggtgcactacaccgggatgcttgaagatggaaagaaaatggattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagataa

SEQ ID NO: 2 - protein, 483 aa, Aequoria Victoria and Human
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRAQASNSAVDGTAGPGSTGSRGVQVETISPGDGRTFPKRGQTCVVHYTGMLEDGKKMDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKMDSSRDRNKPFKFMLGKQEVIRGWEEGVAQMSVGQRAKLTISPDYAYGATGHPGIIPPHATLVFDVELLKLETR

SEQ ID NO: 3 - DNA, 1107 bp, Aequoria Victoria and Human
  CDS (1)..(1107)
atgggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaagaattctgcagatatccagcacagtggcggccgctcgagtctagaggagtgcaggtggaaaccatctccccgggagacgggcgcaccttccccaagcgcggccagacctgcgtggtgcactacaccgggatgcttgaagatggaaagaaaatggattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggagtgcaggtggaaaccatctccccgggagacgggcgcaccttccccaagcgcggccagacctgcgtggtgcactacaccgggatgcttgaagatggaaagaaaatggattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagagggcccttcgaaggtaagcctatccctaaccctctcctcggtctcgattctacgcgtaccggtcatcatcaccatcaccattga

SEQ ID NO: 4 - protein, 368 aa, Aequoria Victoria and Human
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEEFCRYPAQWRPLESRGVQVETISPGDGRTFPKRGQTCVVHYTGMLEDGKKMDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKMDSSRDRNKPFKFMLGKQEVIRGWEEGVAQMSVGQRAKLTISPDYAYGATGHPGIIPPHATLVFDVELLKLETRGPFEGKPIPNPLLGLDSTRTGHHHHHH

SEQ ID NO: 5 - DNA, 1092 bp, Aequoria Victoria and Human
  CDS (1)..(1092)
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctagtgaccaccctgtcttacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctcctagggttcgtgaccgccgccgggatcactctcggcatggacgagctgtacaagtccggactcagatctcgaatcacaagtttgtacaaaaaagcaggctccatgggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatga

SEQ ID NO: 6 - protein, 363 aa, Aequoria Victoria and Human
MVSKGEELFTGVVPILVELDGDVNGHKFSVSGEGEGDATYGKLTLKFICTTGKLPVPWPTLVTTLSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLGFVTAAGITLGMDELYKSGLRSRITSLYKKAGSMGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 7 - DNA, 744 bp, Aequoria Victoria and Human
  CDS (1)..(744)
atggaccattatgattctcagcaaaccaacgattacatgcagccagaagaggactgggaccgggacctgctcctggacccggcctgggagaagcagcagagaaagacattcacggcatggtgtaactcccacctccggaaggcggggacacagatcgagaacatcgaagaggacttccgggatggcctgaagctcatgctgctgctggaggtcatctcaggtgaacgcttggccaagccagagcgaggcaagatgagagtgcacaagatctccaacgtcaacaaggccctggatttcatagccagcaaaggcgtcaaactggtgtccatcggagccgaagaaatcgtggatgggaatgtgaagatgaccctgggcatgatctggaccatcatcctgcgcggatctcgagctcaagcttcgaattctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagttatccgtacgacgtaccagactacgcataa

SEQ ID NO: 8 - protein, 247 aa, Aequoria Victoria and Human
MDHYDSQQTNDYMQPEEDWDRDLLLDPAWEKQQRKTFTAWCNSHLRKAGTQIENIEEDFRDGLKLMLLLEVISGERLAKPERGKMRVHKISNVNKALDFIASKGVKLVSIGAEEIVDGNVKMTLGMIWTIILRGSRAQASNSRILWHEMWHEGLEEASRLYFGERNVKGMFEVLEPLHAMMERGPQTLKETSFNQAYGRDLMEAQEWCRKYMKSGNVKDLLQAWDLYYHVFRRISKTSYPYDVPDYA

SEQ ID NO: 9 - DNA, 723 bp, Aequoria Victoria and Human
  CDS (1)..(723)
atgccagagccagcgaagtctgctcccgccccgaagaagggctccaagaaggcagtgaccaaagcgcagaagaaagatggcaagaagcgcaagcgcagccgcaaggagagttactctgtgtacgtgtacaaggtgctgaaacaggtccatcccgacactggcatctcttccaaggccatgggcatcatgaattctttcgttaacgacatatttgagcgcatcgcgggcgaggcttcccgcctggcgcattacaacaagcgctcgaccatcacctccagggagatccagacggccgtgcgcctgctgcttcccggagagctggccaagcacgccgtgtcggagggcaccaaggccgtcaccaagtacaccagcgctaagagatctcgagctcaagcttcgaattctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagttatccgtacgacgtaccagactacgcataa

SEQ ID NO: 10 - protein, 240 aa, Aequoria Victoria and Human
MPEPAKSAPAPKKGSKKAVTKAQKKDGKKRKRSRKESYSVYVYKVLKQVHPDTGISSKAMGIMNSFVNDIFERIAGEASRLAHYNKRSTITSREIQTAVRLLLPGELAKHAVSEGTKAVTKYTSAKRSRAQASNSRILWHEMWHEGLEEASRLYFGERNVKGMFEVLEPLHAMMERGPQTLKETSFNQAYGRDLMEAQEWCRKYMKSGNVKDLLQAWDLYYHVFRRISKTSYPYDVPDYA

SEQ ID NO: 11 - DNA, 387 bp, Aequoria Victoria and Human
  CDS (1)..(387)
atgggatccaacaagagcaagcccaaggatgccagccagcggagatctcgagctcaagcttcgaattctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagttatccgtacgacgtaccagactacgcataa

SEQ ID NO: 12 - protein, 128 aa, Aequoria Victoria and Human
MGSNKSKPKDASQRRSRAQASNSRILWHEMWHEGLEEASRLYFGERNVKGMFEVLEPLHAMMERGPQTLKETSFNQAYGRDLMEAQEWCRKYMKSGNVKDLLQAWDLYYHVFRRISKTSYPYDVPDYA